(19) United States
(12) Patent Application Publication
(10) Pub. No.: US 2021/0275661 A1
(43) Pub. Date: Sep. 9, 2021

(54) Vaccine

(71) Applicant: VyVax, AB, Tampa, FL (US)

(72) Inventors: Kim Andrea Blom Nihlen, Hagersten (SE); Hans-Gustaf Einar Ljundggren, Stockholm (SE)

(73) Assignee: VyVax AB, Tampa, FL (US)

(21) Appl. No.: 16/494,211

(22) PCT Filed: Mar. 14, 2018
(86) PCT No.: PCT/EP2018/056444

§ 371 Date: Sep. 13, 2019

(30) Foreign Application Priority Data
Mar. 15, 2017 (GB) 1704126.0

(51) Int. Cl. A61K 39/12; A61K 39/39; A61P 31/14

(52) U.S. Cl. CPC A61K 39/12; A61K 2039/58; A61P 31/14; A61K 39/39

(57) ABSTRACT

The invention provides a vaccine composition comprising a Yellow Fever Virus vaccine, for use in vaccinating an individual against infection by a Flavivirus; wherein the Flavivirus is not Yellow Fever Virus. The invention also provides a vaccine composition comprising a Yellow Fever Virus vaccine and one or more additional vaccine against a Flavivirus, for use in vaccinating an individual against infection by the Flavivirus; wherein the Flavivirus is not Yellow Fever Virus.

This application is a 371 application of Patent Cooperation Treaty application number PCT/EP2018/056444 filed on Mar. 14, 2018 which claims priority to United Kingdom patent application number 1704126.0 filed on Mar. 15, 2017, the contents of which are incorporated by reference. All references cited herein are incorporated by reference.

The invention relates generally to vaccines compositions, and in particular to a vaccine composition for use in vaccinating an individual against infection by a Flavivirus.

The listing or discussion of an apparently prior published document in this specification should not necessarily be taken as an acknowledgement that the document is part of the state of the art or is common general knowledge.

Flaviviruses belongs to the family of Flaviviridae and comprise over 70 viruses that cause severe diseases. These viruses are responsible for hundreds of thousands of deaths annually and additional significant morbidity. Most of the viruses are transmitted to vertebrate hosts either by mosquitoes or ticks. Several members of the flavivirus genus, such as dengue virus (DENV), yellow fever virus (YFV), West Nile virus (WNV), Tick-Borne Encephalitis virus (TBEV), Zika virus (ZIKV), and Japanese encephalitis virus (JEV), are highly pathogenic to humans and constitute major international health problems.

Flaviviruses have a single stranded RNA genome that encodes ten proteins (C, prM, E, NS1, NS2A, NS2B, NS3, NS4A, NS4B and NS5), where NS3 and NS5 proteins are considered conserved regions within the 1-5 viral strains of each virus. They can be broadly grouped into viruses that cause vascular leak and haemorrhagic fever (DENV, YFV), and those that cause encephalitis (WNV, ZIKV, TBEV and JEV). Currently, it is not possible to predict which individuals will develop severe clinical illness, and our understanding of the pathogenesis of the diseases caused by the viruses is far from adequate. One common feature of Flavivirus infections is that no specific treatment or effective antiviral drug exists. Some of the infections can, however, be prevented by vaccination. There are currently commercially available vaccines against three Flaviviruses; YFV, JEV and TBEV (see Table 1 below). Of these vaccines, the live attenuated YFV vaccine is one of the most effective and used vaccines on earth.

The live attenuated YFV vaccine was developed in the 1930s and has, in recent years, received renewed attention due to its very high efficacy (a single dose giving protection for at least 10 years) (see Table 1). The YFV vaccine can be used to study human immune responses in a replication-competent viral infection. Adaptive and innate immune responses elicited by the YFV vaccine in humans have been well-characterised.

JEV and TBEV vaccines are based on inactivated viruses (Table 1), with subsequent boosters required to maintain protection.

TBEV is transmitted to humans mainly through infected ticks, and it is estimated that one third of infected individuals develop clinical disease (Tick borne encephalitis, TBE). TBE is a biphasic disease where more than a third of patients will suffer from life-long persistent complications after the acute phase of the infection, including neuropsychiatric symptoms, severe headaches, and a decreased quality of life¹³. Numbers of reported cases have increased rapidly over the last decades in both Europe and Asia^{4,14}, and the infection can only be treated symptomatically as no specific treatment exists. Despite existing vaccines, over two hundred cases of TBE are reported each year in Sweden. Two formalin-inactivated virus based vaccines are available in Europe (FSME-IMMUN, Pfizer Innovations and Encepur, GlaxoSmithKline). These vaccines require booster doses every 3-5 years after the initial vaccinations to maintain protection.

JEV is, as the YFV, transmitted to humans through mosquitoes and is the major cause of epidemic encephalitis in Asia¹. The virus causes Japanese encephalitis (JE) that has a fatality rate as high as 30% among persons with symptomatic disease, and approximately 50% of survivors suffer long-lasting neuropsychiatric sequel. IXIARO® is an inactivated vaccine against JEV, grown in Vero cells. Booster doses are recommended if a person received the two-dose primary vaccination over a year ago, and there is still a continued risk for JEV infection. Further, due to the cost of the vaccine it has been beyond reach of millions of people in South East Asia. Even in western countries, many travelers to endemic areas abstain from vaccination due to the high cost.

There are increasing numbers of TBE vaccine failures reported, and despite a complete JE vaccine program with booster doses, there is still also risk for JEV vaccine failure. Hence, both JEV and TBE vaccines lack the efficacy provided by the YFV vaccine. Improving the JEV and TBE vaccines is of great importance for public health in Sweden, the rest of Europe, and Asia, where these viruses are endemic.

Thus, there is a demand to develop further vaccines to protect against infection with Flaviviruses, and particularly vaccines to protect against infection with Flaviviruses other than YFV.

The inventors have now found that the YFV vaccine generates a cross reactive immune response (particularly, a T cell cross-reactive response) to Flaviviruses other than YFV, including TBEV and ZIKV. Additionally, cross-reactive antibody responses are observed. The inventors have also identified a surprising synergistic effect in terms of the immune response generated against a Flavivirus when a YFV vaccine is used in combination with another Flavivirus vaccine.

The inventors' findings are surprising, because it has not previously been observed that a Flavivirus vaccine can generate a cross-reactive T cells response (Kayser et al., Human Antibody Responses to Immunization With 17D Yellow Fever and Inactivated TBE Vaccine, J Medical Virol., 17:35-45, 1985; Theiler, J Casals: The serological reactions in yellow fever. Am J Trop Med Hyg. 7:585-594 1958; Weyer, Rupprech and Nel: Cross-protective and cross-reactive immune responses to recombinant vaccinia viruses expressing full-length lyssavirus glycoprotein genes, Epidemiol Infect 2008; Turtle, L. et al. Cellular Immune Responses to Live Attenuated Japanese Encephalitis (JE) Vaccine SA14-14-2 in Adults in a JE/Dengue Co-Endemic Area. PLoS Negl Trop Dis 11, e0005263, doi:10.1371/journal.pntd.0005263 (2017)).

Accordingly, a first aspect of the invention provides a vaccine composition comprising a YFV vaccine, for use in vaccinating an individual against infection by a Flavivirus; wherein the Flavivirus is not YFV.

The invention includes the use of a vaccine composition comprising a YFV vaccine, in the manufacture of a medicament for vaccinating an individual against infection by a Flavivirus, wherein the Flavivirus is not YFV.

The invention also includes a method for vaccinating an individual against infection by a Flavivirus, the method comprising the step of administering to the individual a vaccine composition comprising a YFV vaccine; wherein the Flavivirus is not YFV.

By a YFV vaccine we include the meaning of an immunogen that when administered to an individual (eg one who is not immunocompromised or immunosuppressed), is capable of inducing a protective immune response against YFV in the individual. Thus, the vaccine may be one that is protective against a challenge (eg a subsequent or later challenge) by YFV itself, for example by either preventing infection altogether, or by lessening the impact of that infection by decreasing one or more disease symptoms that would otherwise occur, had the vaccine not been administered to the individual.

As used herein, “an immune response” is meant to encompass cellular and/or humoral immune responses that are sufficient to inhibit or prevent infection, or prevent or inhibit disease symptoms caused by the infection. Innate and/or adaptive immune responses are also included.

Whether or not a YFV vaccine is capable of inducing a protective immune response can be determined by any suitable method in the art.

In one embodiment, whether or not a YFV vaccine is capable of inducing a protective immune response is determined by assessing the presence of the YFV in an individual following infection with YFV. If a protective immune response has been induced, the viral load of YFV would be expected to be less at any given time after infection than the viral load of YFV if a protective immune response had not been induced. Typically, the viral load will be reduced by at least 10%, 20%, 30%, 40%, or 50% and more typically at least 60%, 70%, 80%, 90% or 95% compared to the viral load in an individual where no protective immune response has been induced (eg in an individual who has not been administered the YFV vaccine).

Methods for measuring viral load are well known in the art and include both direct and indirect methods. Directly assessing the presence of YFV in an individual may involve directly assessing the presence of the viral genome (eg by reverse transcription polymerase chain reaction) and/or another constituent of the virus such as a viral protein. Another direct approach is the isolation of the virus from blood plasma and its growth in cell culture. Alternatively, viral load can be assessed indirectly. Indirect detection methods typically make use of the fact that, in later stages of infection, the humoral immune response in the form of IgM and IgG antibodies is well established. Viral load can therefore be assessed indirectly by detecting antibodies targeting the infecting virus using, for example enzyme-linked immunosorbent assay techniques. The preferred tool for determining viral load of YFV is detection of virus RNA or viral antigens such as the NS1 antigen.

In another embodiment, whether or not a YFV vaccine is capable of inducing a protective immune response is determined by assessing one or more clinical symptoms of a YFV infection in an individual, following infection of that individual with YFV. If a protective immune response has been induced, the one or more clinical symptoms would be expected to be less in number and/or severity, at any given time after infection than the one or more clinical symptoms if a protective immune response had not been induced. Symptoms of YFV are well known in the art and include fever, headache, chills, back pain (eg extreme back pain), fatigue, loss of appetite, muscle pain, nausea and vomiting. Other symptoms associated with a second, toxic phase of the disease include jaundice due to liver damage, abdominal pain, bleeding in the mouth, eyes and gastrointestinal tract, vomit containing blood, kidney failure, hiccups and delirium. It will also be appreciated that assessing one or more clinical symptoms of a YFV infection may include assessing one or more disorders and/or conditions associated with a YFV infection. Given the protection afforded by the YFV vaccine, it is appreciated that vaccinated individuals would be overrepresented in the asymptomatic population.

In yet another embodiment, whether or not a YFV vaccine is capable of inducing a protective immune response is determined by detecting one or more indicators of that immune response directly in the individual, following infection of the individual with YFV vaccine. By “one or more indicators of an immune response”, we include the meaning of one or more cells and/or molecules and/or genes that are responsible for mediating that immune response and so whose presence or modulation (eg upregulation) can be used to detect the response. The one or more indicators may be indicators of the innate immune response to YFV and/or the adaptive immune response to YFV. The one or more indicators of the adaptive immune response may be indicators of the cellular immune response to YFV and/or the humoral immune response to YFV. By one or more indicators of the immune response, we include the meaning of antibodies that bind specifically to YFV, T cells specific for YFV (eg CD4 and/or CD8 T cells and/or other T cell receptor positive cells including Treg T cells, NK T cells and mucosal associated invariant T cells (MAIT)) and B cells specific for YFV (eg memory B cells and/or plasmablasts and/or plasma cells and/or other B cell receptor positive cells).

Typically, the YFV vaccine induces the individual's immune system to produce antibodies which bind specifically to YFV. Primary antibody targets in YFV are the E-protein and NS1. Preferably, the antibody thus produced specifically binds YFV (eg the E-protein or NS1 protein) with a greater affinity than for any other molecule (eg any non-flavivirus derived molecule) in the individual, such as at least 2, or at least 5, or at least 10, or at least 50 times greater affinity than any other molecule (eg any non-flavivirus derived molecule) in the individual. More preferably, the antibody binds YFV (eg the E-protein or NS1 protein) with at least 100, or at least 1,000, or at least 10,000 times greater affinity than for any other molecule (eg any non-flavivirus derived molecule) in the individual. Methods for detecting antibodies are well known in the art and any suitable technique may be used such as ELISA.

It will be appreciated that the YFV vaccine may induce the individual's immune system to produce T cells specific for YFV. The T cells may be CD8 T cells and/or CD4 T cells and/or Treg cells and/or NK T cells and/or MAIT T cells and/or other T cell receptor positive cells. Methods for detecting such cells are well known in the art and are described in the Examples below, and, for example, in Blom et al, 2013 (J Immunol 190: 2150) and Akondy et al, 2009 (J Immunol 183(12): 7919). Conveniently, flow cytometry is used. Thus, in one embodiment, whether or not a YFV vaccine is capable of inducing a protective immune response is determined by assessing whether the individual has T cells specific for YFV, such as any of CD8 T cells, CD4 T cells, Treg T cells, NK T cells, MAIT T cells or other T cell receptor positive cells.

It will likewise be appreciated that the YFV vaccine may induce the individual's immune system to produce B cells specific for YFV. The B cells may be memory B cells, plasma cells, or plasmablasts. Methods for detecting such cells are well known in the art and are described in the Examples below. Conveniently, flow cytometry is used.

Typically, the YFV vaccine is an inactivated or an attenuated vaccine.

By an “inactivated” vaccine we include the meaning that the YFV has been treated in such a way as to eliminate its capacity to cause disease but still retains its ability to evoke protective immunity. The YFV may be killed. Methods for inactivating viruses for use in a vaccine are well known in the art, and include chemical treatment or treatment with UV light.

By an “attenuated” vaccine we include the meaning that the YFV has been selected or otherwise treated in such a way as to greatly diminish its capacity to cause disease but still retains its ability to evoke protective immunity. Methods for attenuating viruses for use in a vaccine are well known in the art, and include mutation or deletion of specific genes which are involved in virulence, thus limiting the pathogenic potential of the virus.

It is appreciated that the YFV vaccine may be a live vaccine. Preferably, the YFV vaccine is a live attenuated vaccine.

In an embodiment, the YFV vaccine comprises one or more immunogens that correspond to one or more protein components of YFV. By a “protein component” we include the meaning of an entire protein, or a portion of a protein. It is appreciated that the protein portion may or may not be post-translationally modified, eg glycosylated. Thus, by a “protein” we also include a post-translationally modified protein such as a glycoprotein. It is further appreciated that the YFV vaccine may comprise a nucleic acid encoding said protein component or portion thereof.

The one or more protein components may be a structural protein and/or a non-structural protein, or a fragment, variant or derivative thereof. Structural proteins of YFV include anchC, prM and E protein. These form the virus together with the packaged RNA molecule and are called capsid (C, 12-14 kDa), membrane (M, and its precursor prM, 18-22 kDa) and envelope (E, 52-54 Kda). Non-structural (NS) proteins of YFV are numbered 1 to 5 in order of synthesis. Three large non-structural proteins have highly conserved sequences amongst Flaviviruses, NS1 (38-41 kDa), NS3 (68-70 kDa) and NS5 (100-103 kDa), and so the protein components may comprise any one or more of NS1, NS3 and NS5, or a fragment, variant or derivative thereof. Other small proteins in YFV include NS2A, NS2B, NS4A and NS4B. It is particularly preferred if the YFV vaccine comprises NS5 or a fragment, variant or derivative thereof.

Typically, the protein component is a non-structural protein, or a fragment, variant or derivative thereof. Thus, the protein component may be NS5, or a fragment, variant or derivative thereof. Likewise, the protein component may be NS3, or a fragment, variant or derivative thereof.

It is preferred if the YFV vaccine comprises NS5 or a fragment, variant or derivative thereof. The amino acid sequences of NS5 proteins from three strains of YFV are provided in the Examples below and so it will be appreciated that the vaccine may comprise any of these particular NS5 proteins, or a fragment, variant or derivative thereof.

By “fragment” of a protein component of YFV such as a non-structural protein or structural protein of YFV, we include the meaning of a portion of the protein component that retains the ability to raise an immune response in an individual to YFV. The fragment may be between 5 and 200 amino acids. Typically, the fragment is at least 5 amino acids (eg 6, 7, 8, 9, or 10 amino acids), such as at least 10, 20, 30, 40, 50, 60, 70, 80, 90 or 100 amino acids. An example of a suitable fragment of NS5 is ETACLSKAY (SEQ ID NO: 1). Other examples of suitable fragments include VLAPYMPDV (SEQ ID NO: 39), YMPDVLEKL (SEQ ID NO: 40), RNSTHEMYY (SEQ ID NO: 41), RVERIKSEY (SEQ ID NO: 42), WFYDNDNPY (SEQ ID NO: 43), RTWHYCGSY (SEQ ID NO: 44), MAMTDTTPF (SEQ ID NO: 45), KVVNRWLFR (SEQ ID NO: 46), RSHAAIGAY (SEQ ID NO: 47), WLGARYLEF (SEQ ID NO: 48), GVEGIGLQY (SEQ ID NO: 49), AAMDGGGFY (SEQ ID NO: 50), YMSPHHKKL (SEQ ID NO: 51), RPAPGGKAY (SEQ ID NO: 52), RPIDDRFGL (SEQ ID NO: 53), YANMWSLMY (SEQ ID NO: 54), YFHKRDMRL (SEQ ID NO: 55), VKKWRDVPY (SEQ ID NO: 56), RTLIGQEKY (SEQ ID NO: 57), RSHAAIGAY (SEQ ID NO: 58), TPFGQQRVF (SEQ ID NO: 59), MWHVTRGAF (SEQ ID NO: 60), SVKEDLVAY (SEQ ID NO: 61), CARRRLRTL (SEQ ID NO: 62), RRRLRTLVL (SEQ ID NO: 63), DVKFHTQAF (SEQ ID NO: 64), AMCHATLTY (SEQ ID NO: 65), RANESATIL (SEQ ID NO: 66), VVVLNRKTF (SEQ ID NO: 67), RVLDCRTAF (SEQ ID NO: 68), SMLLDNMEV (SEQ ID NO: 69).

By “derivative” of a protein component of YFV such as a non-structural protein or structural protein of YFV, we include the meaning of a protein, or portion of a protein, which has been modified from the form in which it is naturally present in that organism, but which retains the ability to raise an immune response in an individual to YFV.

By “derivative” we also include peptides in which one or more of the amino acid residues are chemically modified, before or after the peptide is synthesised, providing that the function of the peptide, namely the production of a specific adaptive immune response (eg production of specific antibodies in vivo), remains substantially unchanged. Such modifications include forming salts with acids or bases, especially physiologically acceptable organic or inorganic acids and bases, forming an ester or amide of a terminal carboxyl group, and attaching amino acid protecting groups such as N-t-butoxycarbonyl. Such modifications may protect the peptide from in vivo metabolism. The peptides may be present as single copies or as multiples, for example tandem repeats. Such tandem or multiple repeats may be sufficiently antigenic themselves to obviate the use of a carrier. It may be advantageous for the peptide to be formed as a loop, with the N-terminal and C-terminal ends joined together, or to add one or more Cys residues to an end to increase antigenicity and/or to allow disulphide bonds to be formed. If the peptide is covalently linked to a carrier, preferably a polypeptide, then the arrangement is preferably such that the peptide of the invention forms a loop.

By “variant” of a protein component of YFV such as a non-structural protein or structural protein of YFV, we include the meaning of a sequence variant of the protein component or portion thereof which can be used to raise an immune response in an individual to YFV. For example, the protein component of YFV may contain one or more amino acid substitutions compared to the amino acid sequence of the protein component that occurs naturally in nature. Preferably, the variant has at least 60% sequence identity to the native protein component of YFV or portion thereof, such as at least 65%, 70%, 75%, 80%, 85%, 90%, or 95% sequence identity. More preferably, the variant has at least 96%, 97%, 98%, or 99% sequence identity to the native protein component of YFV or portion thereof.

The percent sequence identity between two polypeptides may be determined using any suitable computer program, for example the GAP program of the University of Wisconsin Genetic Computing Group and it will be appreciated that percent identity is calculated in relation to polypeptides whose sequence has been aligned optimally. The alignment may alternatively be carried out using the Clustal W program Thompson et al., (1994) Nucleic Acids Res 22, 4673-80). The parameters used may be as follows: Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

Typically, the sequence variant has fewer than 100, or fewer than 50, or fewer than 40, or fewer than 30, or fewer than 20 amino acid residues different from the native sequence of that protein or portion thereof. More preferably, the sequence variant has 15 or 14 or 13 or 12 or 11 or 10 or 9 or 8 or 7 or 6 or 5 or 4 or 3 or 2 or only 1 amino acid residues different from the native sequence of that protein or portion thereof.

The sequence of the derivative may have been altered to enhance the immunogenicity of the agent, or it may have no effect on its immunogenicity. For example, the derivative may have had one or more amino acid sequences that are not necessary to immunogenicity removed.

It will be appreciated that protein components may be isolated from cultures of the virus directly. Conveniently, however, proteins are made by expression of a suitable DNA construct encoding the protein using recombinant DNA technology. Suitable techniques for cloning, manipulation, modification and expression of nucleic acids, and purification of expressed proteins, are well known in the art and are described for example in Sambrook (2001) Molecular Cloning A Laboratory Manual. Third Edition. Cold Spring Harbor Laboratory Press, Cold Spring Harbor, N.Y.

Alternatively, proteins may be made using protein chemistry techniques for example using partial proteolysis of isolated proteins (either exolytically or endolytically), or by de novo synthesis. Peptides may be synthesised by the Fmoc-polyamide mode of solid-phase peptide synthesis as disclosed by Lu et al (1981) J. Org. Chem. 46, 3433 and references therein.

It will be appreciated that the YFV vaccine may contain an agent to stimulate or enhance stimulation of the immune system. Thus, the vaccine may comprise an adjuvant. However, it is also appreciated that the YFV vaccine itself, by viral replication and uptake by the immune system may serve as an adjuvant. Hence, the YFV vaccine may contain only virus and a carrier (eg physiological solution).

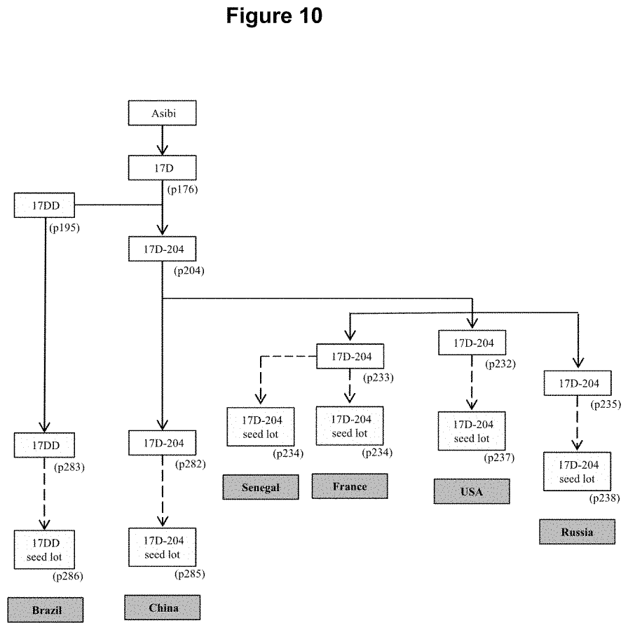

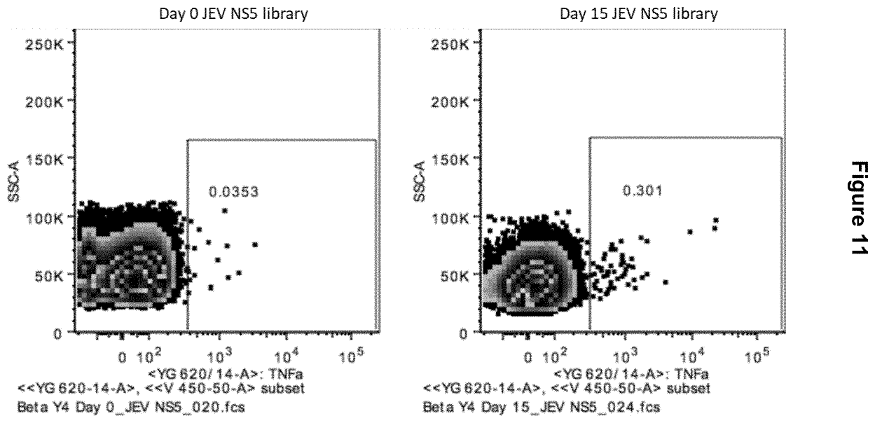

In a preferred embodiment, the YFV vaccine comprises a Yellow Fever virus derived from the Asibi strain isolated from a man named Asibi by passage in Rhesus monkeys (Stokes, 1928, J Am Med Assoc 90: 253). Viral strains derived from the Asibi strain are depicted in FIG. 10, and so it will be appreciated that the YFV vaccine may comprise any of these strains. In a particularly preferred embodiment, the YFV vaccine comprises a viral strain derived from the 17D strain. Preferably, the YFV vaccine is a live attenuated vaccine comprising such a strain.

The 17D vaccine was developed in 1937 and was obtained by 176 passages of Asibi strain in chicken embryo tissue. Currently, substrains of 17D, 17DD and 17D-204 are used for vaccine manufacturing. To date, four vaccine products have been prequalified by the WHO, all of which are commercially available. The vaccines are manufactured by Federal State Unitary Enterprise of Chumakov Institute of Poliomyelitis and Viral Encephalitiides of Russion Acad Med Sci, Institut Pasteur de Dakar, Sanofi Pasteur SA and Bio-Manguinhos/Fiocruz, respectively. Any of these commercially available vaccines may be used in the present invention. Suitable YFV viral strains for vaccine use include:
1. Yellow fever Asibi strain (origin isolate and precursor of all 17D generated vaccines) https://www.ncbi.nlm.nih.gov/nuccore/AY640589.1
2. YFV 17DD (YF-VAX) https://www.ncbi.nlm.nih.gov/nuccore/70724977
3. YFV 17D204 (Stamaril) https://www.ncbi.nlm.nih.qov/nuccore/KF769015.1

In a particularly preferred embodiment, the YFV vaccine is Stamaril® (Sanofi Pasteur SA). Stamaril® contains yellow fever virus 17D-204 strain not less than 1,000 IU Powder: Lactose, Sorbitol E420L-histidine, L-alanine, Sodium chloride, potassium chloride, disodium phosphate, Potassium, Calcium chloride, Magnesium sulfate, Liquid: Sodium chloride, water for injections.

For vaccine use, polynucleotide agents can be delivered in various replicating (e.g. recombinant adenovirus vaccine) or non-replicating (DNA vaccine) vectors.

A typical dose of a vaccine comprised of recombinant protein is about 5-10 μg. A typical dose of a bacterial vaccine is 10⁸ colony forming units per ml.

Typically, the vaccine composition further comprises a pharmaceutically acceptable carrier, diluent or adjuvant. Carriers and adjuvants are well known in the art. Suitable adjuvants include Freund's complete or incomplete adjuvant, muramyl dipeptide, the “Iscoms” of EP 109 942, EP 180 564 and EP 231 039, aluminium hydroxide, saponin, DEAE-dextran, neutral oils (such as miglyol), vegetable oils (such as arachis oil), liposomes, Pluronic® polyols or the Ribi adjuvant system (see, for example GB-A-2 189 141).

The carrier(s) must be “acceptable” in the sense of being compatible with the agent(s) of the invention and not deleterious to the recipients thereof. Typically, the carriers will be water or saline which will be sterile and pyrogen free.

Typically, the vaccine will be administered via transcutaneous, epidermal, intradermal, subcutaneous, intramuscular, intravenous, intraperitoneal, intranasal, oral, pulmonary or other mucosal routes such as eg vaginal or rectal.

The vaccine composition may be formulated for parenteral administration, and may include aqueous or non-aqueous sterile injection solutions which may contain anti-oxidants, buffers, bacteriostats and solutes which render the formulation isotonic with the blood of the intended recipient; and/or aqueous or non-aqueous sterile suspensions which may include suspending agents and thickening agents.

The vaccine composition may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets.

The vaccine composition may be formulated for intranasal administration and may be conveniently delivered in the form of an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the agent(s), e.g. using a mixture of ethanol and the propellant as the solvent, which may additionally contain a lubricant, e.g. sorbitan trioleate.

It is preferred if the YFV vaccine is Stamaril®, which is administered at a dose of 0.5 mL containing at least 4.74 log 10 plaque forming units (PFU).

The efficacy of a vaccine is the percentage reduction of disease in a vaccinated group of people compared to an unvaccinated group, using the most favourable conditions (Weinburg & Szilagyi, 2010, Journal of Infectious Diseases 201(11): 1607-1610). Vaccine efficacy shows how effective the vaccine could be given ideal circumstances and 100% vaccine uptake whereas vaccine effectiveness measures how well a vaccine performs when it is used in routine circumstances in the community. The outcome data (vaccine efficacy) generally are expressed as a proportionate reduction in disease attack rate (AR) between the unvaccinated (ARU) and vaccinated (ARV) studies, and can calculated from the relative risk (RR) of disease among the vaccinated group with use of the following formula: VE=[(ARU−ARV)/ARU]×100, where VE=vaccine efficacy; ARU=attack rate of unvaccinated people; and ARV=attack rate of vaccinated people. Vaccines are never usually 100% protective, although the commercially available YFV vaccines are close (˜typically 99% of vaccinated). Thus, in an embodiment, the YFV vaccine has a VE of at least 50% such as at least 60%, 70%, 80%, 90%, 91%, 92%, 93%, 94%, 95%, 96%, 97%, or 98%, and most preferably the YFV vaccine has a VE of at least 99%.

In an embodiment, the YFV vaccine vaccinates an individual against infection by a Flavivirus, wherein the Flavivirus is one or more selected from the group consisting of: Zika virus; Tick-Bourne Encephalitis Virus; Japanese Encephalitis Virus; West Nile Virus; Saint Louis Encephalitis Virus; and Omsk Haemorrhagic Fever Virus. It is particularly preferred if the Flavivirus is selected from the group consisting of Tick-Borne Encephalitis Virus, Japanese encephalitis virus, and Zika virus.

Dengue virus exists as four serotypes, DENV1-DENV4, and following a primary infection, individuals produce DENV-specific antibodies that bind only to the serotype of infection. Given the apparent lack of cross-reactivity between DENV serotypes, the effect of the YFV vaccine on immunity against the DENV is unknown. Thus, in an embodiment, the Flavivirus is not Dengue Virus. In other words, in one embodiment, the invention provides a method of vaccinating against infection by a Flavivirus, the method comprising the step of administering to the individual a vaccine composition comprising a YFV vaccine; wherein the Flavivirus is not YFV and wherein the Flavivirus is not Dengue Virus. Similarly, in one embodiment, the invention provides a vaccine composition comprising a YFV vaccine for use in vaccinating an individual against infection by a Flavivirus wherein the Flavivirus is not YFV and wherein the Flavivirus is not Dengue Virus. Likewise, in one embodiment, the invention provides a use of a vaccine composition comprising a YFV vaccine in the manufacture of a medicament for vaccinating an individual against infection by a Flavivirus, wherein the Flavivirus is not YFV and wherein the Flavivirus is not Dengue Virus.

By “vaccinating an individual against infection by a Flavivirus”, we include the meaning that protective immunity against the Flavivirus is induced in the individual. For example, the vaccine may prevent and/or reduce a subsequent infection by the Flavivirus in the individual. The vaccine may generate and/or increase immunity in the individual against the Flavivirus.

In one embodiment, whether or not a YFV vaccine is capable of inducing protective immune response against the Flavivirus is determined by assessing the presence of the Flavivirus in an individual following infection with the Flavivirus. If a protective immune response has been induced, the viral load of the Flavivirus would be expected to be less at any given time after infection than the viral load of the Flavivirus if a protective immune response induced by the YFV vaccine had not been induced. Typically, the viral load will be reduced by at least 10%, 20%, 30%, 40%, or 50% and more typically at least 60%, 70%, 80%, 90% or 95% compared to the viral load in an individual where the YFV vaccine did not induce a protective immune response (eg in an individual who has not been administered the YFV vaccine). Methods for measuring viral load are well known in the art and include both direct and indirect methods as described above.

In another embodiment, whether or not a YFV vaccine is capable of inducing a protective immune response against the Flavivirus is determined by assessing one or more clinical symptoms of a Flavivirus infection in an individual, following infection of that individual with the Flavivirus. If the YFV vaccine induces a protective immune response, the one or more clinical symptoms would be expected to be less in number and/or severity, at any given time after infection than the one or more clinical symptoms if the YFV does not induce a protective immune response. It will also be appreciated that where the Flavivirus infection is associated with another disorder and/or condition, then whether or not a YFV vaccine is capable of inducing a protective immune response against the Flavivirus may be determined by assessing whether or not the YFV vaccine reduces the symptoms of that other disorder and/or condition.

Methods for diagnosing infection by particular Flaviviruses and the symptoms of the infections/associated disorders or conditions are outlined below. It will be appreciated these methods may be used to assess whether or not a YFV vaccine is capable of inducing a protective immune response against the Flavivirus, as described herein.

### Zika Virus

Diagnosis: ZI KV-specific IgM antibodies can be detected by ELISA or immunofluorescence assays in serum specimens from day 5 after the onset of symptoms.

Symptoms: Zika virus usually causes a mild infection with symptoms lasting for several days to a week. People usually don't get sick enough to need hospital care, and they very rarely die of the Zika virus infection. For this reason, many people might not realize they have been infected. Symptoms of Zika are similar to other viruses spread through mosquito bites, like Dengue and Chikungunya. Many people infected with Zika virus won't have symptoms or will only have mild symptoms. The most common symptoms of Zika are fever, rash, joint pain, conjunctivitis (red eyes), muscle pain, headache.

Infection related syndromes/conditions: Guillain-Barré syndrome. Birth defects: Microcephaly and congenital Zika syndrome

### Tick-Borne Encephalitis Virus

Diagnosis: During the first phase of the disease, the most common laboratory abnormalities are a low white blood cell count (leukopenia) and a low platelet count (thrombocytopenia). Liver enzymes in the serum may also be mildly elevated. After the onset of neurologic disease, referred to as the second phase, an increase in the number of white blood cells in the blood and the cerebrospinal fluid (CSF) is usually found. Virus can be isolated from the blood during the first phase of the disease. Laboratory diagnosis usually depends on detection of specific IgM or IgG in either blood or CSF, usually appearing later, during the second phase of the disease.

Symptoms: Asymptomatic infection occurs in 60-70% of infected individuals. Infected patients may experience a clinical illness that involves the central nervous system with symptoms of meningitis (e.g., fever, headache, and a stiff neck), encephalitis (e.g., drowsiness, confusion, sensory disturbances, and/or motor abnormalities such as paralysis), or meningoencephalitis. Long lasting (sometimes lifelong) symptoms occurs in over 30% of patients being hospitalized.

### Japanese Encephalitis Virus

Diagnosis: Laboratory diagnosis of JE is generally accomplished by testing of serum or cerebrospinal fluid (CSF) to detect virus-specific IgM antibodies. JE virus IgM antibodies are usually detectable 3 to 8 days after onset of illness and persist for 30 to 90 days, but longer persistence has been documented. Therefore, positive IgM antibodies occasionally may reflect a past infection or vaccination.

Symptoms: Less than 1% of people infected with Japanese encephalitis (JE) virus develop clinical illness. In persons who develop symptoms, the incubation period (time from infection until illness) is typically 5-15 days. Initial symptoms often include fever, headache, and vomiting. Mental status changes, neurologic symptoms, weakness, and movement disorders might develop over the next few days. Seizures are common, especially among children.

### West Nile Virus

Diagnosis: Routine clinical laboratory studies do not distinguish WNV infection from many other viral infections. Patients with neuroinvasive disease generally have lymphocytic pleocytosis in the cerebrospinal fluid (CSF), but neutrophils may predominate early in the course of illness. Detection of WNV-specific immunoglobulin (Ig) M in serum or CSF provides strong evidence of recent WNV infection. In most patients, IgM antibody against WNV is usually detectable by 8 days after illness onset; however, in patients with WNV neuroinvasive disease, specific IgM is almost always detectable in serum and CSF by the time of symptom onset.

Symptoms: About 80% of human infections are apparently asymptomatic. Of those persons in whom symptoms develop, most have self-limited West Nile fever (WNF), characterized by the acute onset of fever, headache, fatigue, malaise, muscle pain, and weakness; gastrointestinal symptoms and a transient macular rash on the trunk and extremities are sometimes reported. Neuroinvasive disease develops in <1% of WNV-infected persons, for example, in such forms as meningitis, encephalitis, or paralysis (the proportion of reported cases that are neuroinvasive disease is higher because neuroinvasive disease is more likely to be reported than WNF or asymptomatic infections). The risk for encephalitis increases with age.

### Saint Louis Encephalitis Virus

Diagnosis: SLEV disease can be made by the detection of SLEV-specific IgM antibody in serum or CSF. A positive SLEV IgM test result should be confirmed by neutralizing antibody testing of acute- and convalescent-phase serum specimens at a state public health laboratory or CDC.

Symptoms: Less than 1% of St. Louis encephalitis virus (SLEV) infections are clinically apparent and the vast majority of infections remain undiagnosed. The incubation period for SLEV disease (the time from infected mosquito bite to onset of illness) ranges from 5 to 15 days. Onset of illness is usually abrupt, with fever, headache, dizziness, nausea, and malaise. Signs and symptoms intensify over a period of several days to a week. Some patients spontaneously recover after this period; others develop signs of central nervous system infections, including stiff neck, confusion, disorientation, dizziness, tremors and unsteadiness. Coma can develop in severe cases. The disease is generally milder in children than in older adults. About 40% of children and young adults with SLEV disease develop only fever and headache or aseptic meningitis; almost 90% of elderly persons with SLEV disease develop encephalitis. The overall case-fatality ratio is 5 to 15%. The risk of fatal disease also increases with age. In acute SLEV neuroinvasive disease cases, cerebrospinal fluid (CSF) examination shows a moderate (typically lymphocytic) pleocytosis. CSF protein is elevated in about a half to two-thirds of cases. Computed tomography (CT) brain scans are usually normal; electroencephalographic (EEG) results often show generalized slowing without focal activity.

### Omsk Haemorrhagic Fever Virus

Diagnosis: Serology, IgM IgG

Symptoms: After an incubation period of 3-8 days, the symptoms of OHF begin suddenly with chills, fever, headache, and severe muscle pain with vomiting, gastrointestinal symptoms and bleeding problems occurring 3-4 days after initial symptom onset. Patients may experience abnormally low blood pressure and low platelet, red blood cell, and white blood cell counts. After 2 weeks, some patients may recover, although others might not. They might experience a focal hemorrhage in mucosa of gingival, uterus and lungs, and occasional neurological involvement. If the patient still has OHF after 3 weeks, then a second wave of symptoms will occur. It also includes signs of encephalitis. In most cases if the sickness does not fade away after this period, the patient will die. Patients that recover from OHF may experience hearing loss, hair loss, and behavioural or psychological difficulties associated with neurological conditions.

It will be understood that vaccinating the individual with the YFV vaccine may generate and/or increase immunity in the individual against the Flavivirus. The immunity may be innate and/or adaptive immunity. The immunity may be cellular and/or humoral immunity.

In one embodiment, vaccinating the individual with the YFV vaccine generates and/or increases cellular immunity against the Flavivirus which comprises T cell activity in the individual against the Flavivirus. For example, the vaccine may generate new T cells and/or increase the level of T cells that recognise YFV and which are cross-reactive to the other Flavivirus. Thus, it will be appreciated that the vaccine composition for use according to the first aspect of the invention may generate and/or increase the number of cross-reactive T cells, ie T cells that recognise YFV but which also recognise another Flavivirus which is not YFV. Where the YFV vaccine increases cellular immunity against the Flavivirus which comprises T cell activity in the individual against the Flavivirus, the cellular immunity is typically increased by at least 2 fold, 3 fold, 4 fold, 5 fold, or 10 fold.

T cells leave the thymus as immature, naive T cells. The engagement and binding of the CD3/TCR complex to a peptide-loaded MHC molecule (in this case vaccine derived peptide) will activate a naive T cell, but efficient activation requires co-stimulation through dedicated co-stimulatory receptors. If the T cell does not receive the second co-stimulatory signal, it will undergo apoptosis. This activation, or priming, which occurs via interactions with a professional antigen presenting cell (APC), will allow the T cell to mature, divide and differentiate into effector and memory cells. A typical co-receptor, found on the surface of the T cell is CD28, which binds to CD80 or CD86 on the APC to initiate activation. When a CD8 T cell becomes activated, it proliferates and this clonal expansion is supported by the T cell growth factor IL-2. The T cell differentiates into subsets of effector cells, many of which enter the blood and migrate to sites of infection. Following clearance of a pathogen, the effector T cell population contracts and the majority of the pathogen-specific T cells enter apoptosis. A small pool of pathogen specific T cells (5-10%), however, survives as memory cells. The pool of memory cells is maintained in a cytokine-dependent manner mainly through the actions of IL-7 and IL-15 to promote memory T cell survival, independently of specific antigen. Upon re-exposure to the pathogen, the memory cells quickly expand to large numbers of effector T cells. Memory T cell subsets were initially defined by the ability to mediate immediate effector functions, and the ability of the cell to migrate to secondary lymphoid organs.

The T cell activity generated in the individual may include the generation of any of CD4, CD8, Treg T cells, NK T cells, MAIT T cells or other T cell receptor positive cells (eg T cells with more innate immune functions) that recognise YFV and which are cross reactive to the other Flavivirus. Measuring populations of T cells is standard practice in the art and typically involves flow cytometry, for example making use of markers that are expressed on particular subsets of T cell.

In one embodiment, administration of the YFV vaccines generates and/or increases the level of CD8 T cells that recognise the epitope ETACLSKAY (SEQ ID NO: 1) in HLA-A1 positive individuals. ETACLSKAY (SEQ ID NO: 1) is an epitope within the NS5 protein of YFV that is also in the NS5 protein of TBEV.

Other cross-reactive T cells that are generated and/or increased following YFV vaccination are expected to be directed at peptides that are shared (or very similar) among different Flavivirus proteins (eg NS5 or NS3 proteins). For example, the T cells may bind to a non-structural protein of the Flavivirus, such as NS5 or NS3. Predicted T cell epitopes for Zika virus and TBE virus are listed in Tables 2 and 3 in the Examples below, and so in one embodiment, the T cells may bind to any of the epitopes listed in Tables 2 and 3 (namely ETACLAKSY (SEQ ID NO: 1). RTWAYHGSY (SEQ ID NO: 2), ALNTFTNLV (SEQ ID NO: 3), YMWLGARFL (SEQ ID NO: 4), RTTWSIHGK (SEQ ID NO: 5), CVYNMMGKR (SEQ ID NO: 6), GLVRVPLSR (SEQ ID NO: 7), YTYQNKVVK (SEQ ID NO: 8), NMMGKREKK (SEQ ID NO: 9), GLGLQRLGY (SEQ ID NO: 10), VPCRHQDEL (SEQ ID NO: 11), GLQRLGYVL (SEQ ID NO: 12), WLGARFLEF (SEQ ID NO: 13), LLYFHRRDL (SEQ ID NO: 14) SGQVVTYAL (SEQ ID NO: 15), STLNGGLFY (SEQ ID NO: 16), ETACLSKAY (SEQ ID NO: 17), GVEGISLNY (SEQ ID NO: 18), VMEWRDVPY (SEQ ID NO: 19), VLAPYRPEV (SEQ ID NO: 20), YMWLGSRFL (SEQ ID NO: 21), MLVSGDDCV (SEQ ID NO: 22), YALNTLTNI (SEQ ID NO: 23), TLTNIKVQL (SEQ ID NO: 24), CVYNMMGKR (SEQ ID NO: 25), KLGEFGVAK (SEQ ID NO: 26), AKVKSNAAL (SEQ ID NO: 27), VVTYALNTL (SEQ ID NO: 28), IAKVKSNAA (SEQ ID NO: 29), SGQVVTYAL (SEQ ID NO: 30)).

Cross-reactivity of a T cell population can be measured using a conventional T cell stimulation assay as is well known in the art, and described in the Examples. Such assays are typically ex vivo where blood samples are taken from donors and processed such that primary cultures of blood cells are used directly in such assays. The methods broadly involve either incubating peptide or proteinaceous samples with a mixture of APCs and T cells prior to measurement of T cell responses, or incubating peptide or proteinaceous samples with APCs and then adding T cells prior to measurement of T cell responses. In both types of assay, multiple blood samples may be used individually for parallel testing of each individual peptide or proteinaceous sample, and T cell responses are then measured usually at a single time point. Thus, whether a particular T cell population is cross-reactive against another Flavivirus may be assessed by stimulating that T cell population with a peptide or proteinaceous sample prepared from that other Flavivirus (eg NS5 protein) and measuring the response. If the peptide or proteinaceous sample generates a response then the T cell population is cross reactive to that Flavivirus. T cell responses are typically measured either by incorporation of a pulse of radioactive label such as tritiated thymidine (3HTdR) into proliferating T cells (“T cell proliferation”) or by release of cytokines such as IFN-g, TNF and/or IL-2 from activated T cells (“cytokine release”). It will also be appreciated that MHC Class I multimers (eg tetramers, pentamers or dextramers) may be used to detect cross-reactive T cells (Wooldridge et al, 2009, Immunology, 126(2): 147-64). The methods described in Blom et al, 2013 (J Immunol 190: 2150) may be used.

In one embodiment, vaccinating the individual with the YFV vaccine generates and/or increases adaptive immunity against the Flavivirus which comprises B cell activity and/or antibody activity in the individual against the Flavivirus.

For example, the vaccine may generate new B cells and/or increase the number of B cells that recognise YFV and which are cross-reactive to the other Flavivirus. Particular types of B cell that may be generated and/or increased by the vaccine are described in more detail below.

Where the YFV vaccine increases adaptive immunity against the Flavivirus which comprises B cell activity and/or antibody activity in the individual against the Flavivirus, the B cell activity and/or antibody activity is typically increased by at least 2 fold, 3 fold, 4 fold, 5 fold, or 10 fold.

Different B cell compartments can be identified according to their phenotype, and several B cell subsets circulate in the blood during the acute phase of an infection. Naïve B cells, memory B cells and plasma cells (PCs) can be phenotyped by staining with surface markers followed by flow cytometry. During a primary infection, naïve B cells are stimulated and develop into antigen-specific B cells. These B cells either differentiate into memory B cells, which reside in the secondary lymphoid organs, or into PCs, which secrete antigen-specific Abs. Prior to differentiation into PCs, B cells undergo several cycles of proliferation and differentiate into an intermediate state called plasmablasts (PBs). Short-lived PCs are active during the acute infection, while long-lived PCs (LLPCs) migrate to the bone marrow and are responsible for long-term humoral immunity. Memory B cells, which retain antigen-specific Abs at their surface, undergo affinity maturation, and only the clones bearing the Abs with the highest affinity survive long-term. Memory B cells are the cells implicated in the antigen recall response and are rapidly activated during a secondary infection.

Thus, the vaccine may generate and/or increase the number of naïve B cells, memory B cells, plasma cells and/or plasmablasts that recognise YFV and which are cross-reactive to the other Flavivirus. The B cells may bind to a non-structural protein of the Flavivirus such as NS5 or NS3.

In an embodiment, the B cells generated and/or increased by the YFV vaccine bind to any of the epitopes ALNTLTNIKVQLIRMME (SEQ ID NO: 31); ALNTLTNIKVQLIRMMEG (SEQ ID NO: 32); GKALYFLNDMAKTRKDIG (SEQ ID NO: 33); or WSIHASGAWMTTEDMLDV (SEQ ID NO: 34). These epitopes are found within the NS5 protein of TBEV (Kuivanen S, Hepojoki J, Vene S, Vaheri A, Vapalahti O. Identification of linear human B-cell epitopes of tick-borne encephalitis virus. Virol J 2014; 11: 115).

In another embodiment, the B cells generated and/or increased by the YFV vaccine bind to the epitope PWLAWHVAANVSSVTDRS (SEQ ID NO: 35). This epitope is found within the NS3 protein of TBEV.

Cross-reactivity of a B cell population can be measured using a conventional B cell stimulation assay as is well known in the art, and described in the Examples. Such assays are typically ex vivo where blood samples are taken from donors and processed such that primary cultures of blood cells are used directly in such assays. The methods broadly involve either incubating peptide or proteinaceous samples with B cells and measuring the B cell response. Thus, whether a particular B cell population is cross-reactive against another Flavivirus may be assessed by stimulating that B cell population with a peptide or proteinaceous sample prepared from that other Flavivirus and measuring the response. If the peptide or proteinaceous sample generates a response then the B cell population is cross reactive to that Flavivirus. B cell responses are typically measured by incorporation of a pulse of radioactive label such as tritiated thymidine (3HTdR) into proliferating B cells (“B cell proliferation”) or by increase of the intracellular expression of the proliferation marker Ki67. Flow cytometry may be used, as is illustrated in the Examples.

The vaccine may generate antibodies that bind to YFV but which also bind to (ie cross react with) the other Flavivirus. Typically, the antibodies include those of the IgG and/or IgM class. Cross reactive antibodies may be detected by standard techniques known in the art, including an enzyme-linked immunospot (ELISPOT) assay (eg for detecting IgM or IgG) (Czerkinsky C, Nilsson L, Nygren H, Ouchterlony O, Tarkowski A (1983). “A solid-phase enzyme-linked immunospot (ELISPOT) assay for enumeration of specific antibody-secreting cells”. J Immunol Methods. 65 (1-2): 109-121) and a plaque reduction neutralisation test (PRNT) assay (Schmidt, N J; J Dennis; E H Lennette (1976-07). “Plaque reduction neutralization test for human cytomegalovirus based upon enhanced uptake of neutral red by virus-infected cells.” Journal of Clinical Microbiology. 4 (1): 61-66) and Vene S, Haglund M, Vapalahti O, Lundkvist A. A rapid fluorescent focus inhibition test for detection of neutralizing antibodies to tick-borne encephalitis virus. J Virol Methods 1998; 73(1): 71-5) (see also Example 1). ELISA may also be used (see also Jolles S, Sewell W A, Misbah S A. Clinical uses of intravenous immunoglobulin. Clin Exp Immunol 2005; 142(1): 1-11).

The basic design of the PRNT assay allows for virus-antibody interaction to occur in a test tube or microtiter plate, and then measuring antibody effects on viral infectivity by plating the mixture on virus-susceptible cells. The cells are overlaid with a semi-solid media that restricts spread of progeny virus. Each virus that initiates a productive infection produces a localized area of infection (a plaque) that can be detected in a variety of ways. Plaques are counted and compared back to the starting concentration of virus to determine the percent reduction in total virus infectivity. In the PRNT assay, the serum specimen being tested is usually subjected to serial dilutions prior to mixing with a standardized amount of virus. The concentration of virus is held constant such that, when added to susceptible cells and overlaid with semi-solid media, individual plaques can be discerned and counted. In this way, PRNT end-point titers can be calculated for each serum specimen at any selected percent reduction of virus activity. For example, a reduction in plaque count of 50% (PRNT₅₀) may be used as the neutralizing end point. This yields a PRNT₅₀ value. Plaques generated by test sera at varying dilutions and the control preparations are counted. The percentage of plaques counted in test sera are compared with the number of plaques from the control preparation. The reciprocal of the lowest dilution of test sera to neutralize 50% of the control virus input represents the PRNT₅₀ titer. Therefore, the PRNT₅₀ titer is calculated by counting plaques and reporting the titer as the reciprocal of the last serum dilution to show 50% reduction of the control plaque count as based on the back-titration of control plaques (Cutchins et al (J Immunol. 1960 September; 85:275-83). It will be appreciated that other neutralizing end points may be used (eg PRNT₈₀ or PRNT₉₀ corresponding to a reduction in plaque count of 80% or 90% respectively).

Typically, the YFV vaccine induces the individual's immune system to produce antibodies which bind specifically to YFV and the other Flavivirus. Primary antibody targets are the E-protein and NS1. Preferably, the antibody thus produced specifically binds YFV and the other Flavivirus (eg the E-protein or NS1 protein thereof) with a greater affinity than for any other molecule (eg any non-flavivirus derived molecule) in the individual, such as at least 2, or at least 5, or at least 10, or at least 50 times greater affinity than any other molecule (eg any non-flavivirus derived molecule) in the individual. More preferably, the antibody binds YFV and the other Flavivirus (eg the E-protein or NS1 protein thereof) with at least 100, or at least 1,000, or at least 10,000 times greater affinity than for any other molecule (eg any non-flavivirus derived molecule) in the individual. Methods for detecting antibodies are well known in the art and any suitable technique may be used such as ELISA.

Without wishing to be bound by any theory, the inventors believe that the YFV vaccine would vaccinate an individual against infection by a Flavivirus regardless of whether or not the individual had been previously infected with that Flavivirus and/or vaccinated against that Flavivirus. For example, previously infected or vaccinated individuals may get a boosting effect with cross reactive YFV specific T cells. Pre-infection with and/or vaccination against other Flaviviruses may increase the protection to the other Flavivirus. Hence, in an embodiment, the individual has previously been infected with the Flavivirus and/or vaccinated against the Flavivirus. Of course, if the individual had not been previously infected with or vaccinated against the Flavivirus, the cross reactive YFV specific T cells generated would be expected to give the first immunity against the Flavivirus in the individual. Thus, in an alternative embodiment, the individual has not previously been infected with the Flavivirus and/or vaccinated against the Flavivirus.

Whether or not an individual has previously been infected with a Flavivirus and/or vaccinated against the Flavivirus can be determined by standard methods known in the art such as any serological assay which detects antibodies, eg Flavivirus specific antibodies.

Without wishing to be bound by any theory, the inventors believe that the YFV vaccine would vaccinate an individual against infection by a Flavivirus regardless of whether or not the individual had been previously infected with YFV and/or vaccinated against YFV. Thus, in one embodiment, the individual has not previously been infected with YFV and/or vaccinated with the YFV vaccine, whereas in an alternative embodiment, the individual has previously been infected with YFV and/or vaccinated with the YFV vaccine. Individuals that have been previously infected with YFV and/or vaccinated against YFV are still expected to benefit from increased immunity against the Flavivirus upon receiving the YFV. The immune system is expected to get a boost through recall responses caused by the YFV vaccine.

Whether or not an individual has previously been infected with YFV and/or vaccinated against YFV can be determined by standard methods known in the art such as any serological assay which detects antibodies, eg YFV specific antibodies.

In an embodiment, the individual has previously been infected with the Flavivirus and/or vaccinated against the Flavivirus, but has not been previously infected with YFV and/or vaccinated against YFV.

In an embodiment, the individual has not previously been infected with the Flavivirus and/or vaccinated against the Flavivirus and has not been previously infected with YFV and/or vaccinated against YFV.

In an embodiment, the individual has previously been infected with the Flavivirus and/or vaccinated against the Flavivirus, and has previously been infected with YFV and/or vaccinated against YFV.

In an embodiment, the individual has not previously been infected with the Flavivirus and/or vaccinated against the Flavivirus and has previously been infected with YFV and/or vaccinated against YFV.

It is preferred if the individual is in a YFV naïve state, ie the individual has not previously been infected with YFV and/or vaccinated against YFV. Without wishing to be bound by any theory, if an individual had had a true YFV infection, the immune system may directly kill off the vaccine and the adjuvant effect would be diminished or lost. However, cross reactive T cells and antibodies may get a boost, thereby inducing protection. Thus, the protective mechanism may differ as compared to YFV naïve individuals.

In a preferred embodiment, the individual has previously been infected with the Flavivirus and/or vaccinated against the Flavivirus, but has not been previously infected with YFV and/or vaccinated against YFV.

In a preferred embodiment, the individual has not previously been infected with the Flavivirus and/or vaccinated against the Flavivirus and has not been previously infected with YFV and/or vaccinated against YFV.

As explained above, the efficacy of a vaccine is the percentage reduction of disease in a vaccinated group of people compared to an unvaccinated group, using the most favourable conditions. The inventors have found that the YFV vaccine provides immunity in an individual against a Flavivirus where that Flavivirus is not the YFV. Typically, the YFV vaccine has a VE in providing immunity against that other Flavivirus of at least 50%, such as at least 55%, 60%, 65%, 70%, 75%, 80%, 85%, 90% or 95%. It is preferred if the YFV vaccine has at least 85% VE. It will be appreciated that the precise VE will depend on host factors (eg host genetics and host immune status), infection dose and likely many other factors), although typically the VE in providing immunity against that other Flavivirus will be above 50%. Measuring VE may make use of any of the techniques described above for assessing the level of immunity to a given Flavivirus, including serology (eg measuring cross-reactive IgG antibodies by ELISA) or by measuring cross-neutralising antibodies using PRNT.

In an embodiment, the immunity against the Flavivirus that is provided by the YFV vaccine (eg the prevention and/or reduction in a subsequent infection by the Flavivirus, or the prevention and/or reduction in one or more disorders and/or conditions associated with infection by the Flavivirus) lasts for a period of up to 1 year, or 5 years or 10 years. Conveniently, this would be measured by assessing the concentration of cross-reactive antibodies against the Flavivirus in the individual using methods known in the art and described above. The measurement would be a measurement of the antibody concentration without a challenge by the Flavivirus. For example, one can measure IgG levels by ELISA and neutralising antibodies by PRNT. Typically, this is done by taking a sample from an individual at a given time point and comparing the levels of cross-reactive antibodies against the Flavivirus, with the levels in positive and/or negative controls. The positive controls may be individuals vaccinated and/or previously infected with YFV or TBE. The negative control may be individuals that have not been vaccinated and/or previously infected with YFV or TBE. Maintained immunity may be considered to be a reading of neutralising antibodies that reduces the number of plaques in a PRNT assay compared to a negative control or maintained titers of IgG that are comparable to IgG levels in positive controls.

As described in more detail in the Examples, it will be appreciated that the number of plaques in a PRNT assay can be used as an indicator of the concentration of neutralising antibody in a serum sample. The higher the titre of neutralising antibody, the greater the reduction in the number of plaques as compared to a negative control (eg virus alone without serum sample). Preferably, the titre of neutralising antibody that is indicative of immunity against the Flavivirus provided by the YFV vaccine, is one that gives at least an 80% reduction of the number of plaques as compared to a negative control, such as at least an 85% or 90% or 95% or 99% or 100% reduction. In one embodiment, the titre of neutralising antibody that is indicative of immunity against the Flavivirus provided by the YFV vaccine is one that gives a PRNT90 value of 5 or more.

In an embodiment, the reduction in a subsequent infection by the Flavivirus and/or the reduction in the one or more disorder and/or condition associated with infection by the Flavivirus, that is afforded by the YFV vaccine, is a reduction by 100%, or by 90%, or by 80%, or by 70%, or by 60%, or by 50%.

It is expected that once an individual has been vaccinated with the YFV vaccine, the viral load of the Flavivirus other than YFV is less at any given time after infection (with the other Flavivirus) than the viral load of YFV if a protective immune response had not been induced. Typically, the viral load will be reduced by at least 10%, 20%, 30%, 40%, or 50% and more typically at least 60%, 70%, 80%, 90% or 95% compared to the viral load in an individual which had not been administered the YFV vaccine. Methods of measuring viral load are well known in the art and include those described above. Viral load may be decreased due to an increase in viral clearance and/or a decrease in viral proliferation (eg arising from the neutralisation of free virus by cross-reactive antibodies and/or viral infected cells being killed by cross-reactive T cells).

A second aspect of the invention provides a vaccine composition comprising a Yellow Fever Virus vaccine and one or more additional vaccine against a Flavivirus, for use in vaccinating an individual against infection by the Flavivirus; wherein the Flavivirus is not Yellow Fever Virus. Thus, it will be appreciated that the vaccine composition contains both a YFV vaccine and one or more additional vaccines against a Flavivirus.

Similarly, the invention includes the use of a vaccine composition comprising a Yellow Fever Virus vaccine and one or more additional vaccine against a Flavivirus, in the manufacture of a medicament for vaccinating an individual against infection by a Flavivirus; wherein the Flavivirus is not Yellow Fever Virus.

Likewise, the invention includes a method for vaccinating an individual against infection by a Flavivirus, the method comprising the step of administering to the individual a vaccine composition comprising a Yellow Fever Virus vaccine and one or more additional vaccine against a Flavivirus; wherein the Flavivirus is not Yellow Fever Virus.

A third aspect of the invention provides a vaccine composition comprising a Yellow Fever Virus vaccine for use in vaccinating an individual against infection by the Flavivirus; wherein the use comprises administering to the individual the Yellow Fever Virus vaccine and the one or more additional vaccine against a Flavivirus; and wherein the Flavivirus is not Yellow Fever Virus. It is appreciated that in this aspect of the invention, the YFV vaccine and the one or more additional vaccine against a Flavivirus need not be part of the same vaccine composition, but may be administered separately as discussed further below.

Similarly, the invention includes the use of a vaccine composition comprising a Yellow Fever Virus vaccine in the manufacture of a medicament for vaccinating an individual against infection by a Flavivirus;

wherein the use comprises administering to the individual the Yellow Fever Virus vaccine and the one or more additional vaccine against a Flavivirus; and wherein the Flavivirus is not Yellow Fever Virus.

Likewise, the invention includes a method for vaccinating an individual against infection by a Flavivirus, the method comprising the steps of administering to the individual a Yellow Fever Virus vaccine and the one or more additional vaccine against a Flavivirus; wherein the Flavivirus is not Yellow Fever Virus.

Preferences for the YFV vaccine and routes and methods of administration include those described above in relation to the first aspect of the invention. It is particularly preferred if the YFV vaccine is Stamaril® or YF-VAX®.

In one embodiment of the third aspect of the invention, the Yellow Fever Virus vaccine and the one or more additional vaccine against a Flavivirus are administered substantially simultaneously to the individual.

In an alternative embodiment of the third aspect of the invention, the Yellow Fever Virus vaccine and the one or more additional vaccine against a Flavivirus are administered sequentially to the individual. Thus, the individual may be one who has already been administered the YFV vaccine, and the one or more additional vaccine against a Flavivirus are subsequently administered to the individual. Alternatively, the individual may be one who has already been administered the one or more additional vaccine against a Flavivirus, and the YFV vaccine is subsequently administered to the individual.

The invention thus includes a vaccine composition comprising a YFV vaccine for use in vaccinating an individual against a Flavivirus, wherein the individual is one who is administered one or more additional vaccine against the Flavivirus, and wherein the Flavivirus is not YFV. The one or more additional vaccine against the Flavivirus may be administered before, at the same time, or after the YFV vaccine.

The invention thus includes the use of a YFV vaccine in the manufacture of a medicament for vaccinating an individual against a Flavivirus, wherein the individual is one who is administered one or more additional vaccine against the Flavivirus, and wherein the Flavivirus is not YFV. The one or more additional vaccine against the Flavivirus may be administered before, at the same time, or after the YFV vaccine.

The invention similarly includes one or more vaccine against a Flavivirus for use in vaccinating an individual against the Flavivirus, wherein the individual is one who is administered a YFV vaccine, and wherein the Flavivirus is not YFV. The YFV vaccine may be administered before, at the same time, or after the one or more vaccine against the Flavivirus.

The invention likewise includes the use of one or more vaccine against a Flavivirus for the manufacture of a medicament for vaccinating an individual against the Flavivirus, wherein the individual is one who is administered a YFV vaccine, and wherein the Flavivirus is not YFV. The YFV vaccine may be administered before, at the same time, or after the one or more vaccine against the Flavivirus.

Where the YFV vaccine and one or more additional vaccine against a Flavivirus are not administered simultaneously, they are administered at least 1 day apart, such as at least 2, 3, 4, 5, 6, or 7 days apart. Viral load peaks 7 days after administration with commonly used YFV vaccines, and so in one embodiment the YFV vaccine and the one or more additional vaccine against a Flavivirus are administered 7 days apart, at least 7 days apart or no more than 7 days apart. Generally, the YFV vaccine and one or more additional vaccine against a Flavivirus are not administered more than 14 days apart, for example no more than 13, 12, 11, 10, 9, 8, or 7 days apart.

In an embodiment, the YFV vaccine is administered 7-14 days before the one or more additional vaccine against a Flavivirus are administered, for example 7, 8, 9, 10, 11, 12, 13 or 14 days before.

In another embodiment, the one or more additional vaccine against a Flavivirus are administered 7-14 days before the YFV vaccine, for example 7, 8, 9, 10, 11, 12, 13, or 14 days before.

It will be appreciated that the YFV vaccine and the one or more additional vaccine against the Flavivirus may be administered simultaneously, for example by the same route of administration, or that the YFV vaccine and the one or more additional vaccine against the Flavivirus may be administered sequentially, for example by separate routes of administration. Suitable methods and routes or administration of the vaccines include those described above in relation to the first aspect of the invention. Typically, the vaccines are administered subcutaneously or intramuscularly, eg in the arm.

By a vaccine against a Flavivirus, we include the meaning of an immunogen that when administered to an individual (eg one who is not immunocompromised or immunosuppressed), is capable of inducing a protective immune response against the Flavivirus in the individual. Thus, the vaccine may be one that is protective against a challenge (eg a subsequent or later challenge) by the Flavivirus itself, for example by either preventing infection altogether, or by lessening the impact of that infection by decreasing one or more disease symptoms that otherwise occur, had the vaccine not been administered to the individual.

Whether or not a vaccine is capable of inducing a protective immune response can be determined by any suitable method in the art, including those described above in relation to the first aspect of the invention. For example, it may be determined by assessing the presence of the Flavivirus in an individual following infection with the Flavivirus (eg by assessing viral load). It may be determined by assessing one or more clinical symptoms of a Flavivirus infection in an individual, following infection of that individual with the Flavivirus. The symptoms of a range of Flavivirus infections are provided above. It may also be determined by detecting one or more indicators of the immune response directly in the individual, following infection of the individual with the Flavivirus (eg the presence of antibodies that specifically bind to the Flavivirus, T cells specific for the Flavivirus, and B cells specific for the Flavivirus). It will be understood that any of the methods described above in relation to determining whether a YFV vaccine is capable of inducing a protective immune response against YFV would likewise be applicable to determining whether a vaccine against a Flavivirus is capable of inducing a protective immune response against that Flavivirus.

Generally, the vaccine against a Flavivirus is an inactivated or an attenuated vaccine, such as a live attenuated vaccine.

In an embodiment of the second and third aspects of the invention, the vaccine against a Flavivirus comprises one or more immunogens that correspond to one or more protein components of the Flavivirus. It is appreciated that the vaccine may comprise a nucleic acid encoding said protein component or portion thereof. Suitable protein components, and portions, variants and derivatives thereof include those described above in relation to the first aspect of the invention. It is preferred if the vaccine includes at least the NS5 protein of the particular Flavivirus.

By one or more vaccine against a Flavivirus, we include the meaning of at least 2, 3, 4, 5, 6, 7, 8, 9, 10 or more vaccines against a Flavivirus.

When two or more vaccines against a Flavivirus are used, it will be appreciated that the two or more vaccines against a Flavivirus against a Flavivirus may be two or more vaccines against the same Flavivirus, or against different Flaviviruses. Thus, the two or more vaccines against a Flavivirus may comprise two or more vaccines against different Flaviviruses respectively, eg 3 or more different Flaviviruses, 4 or more different Flaviviruses, and so on.

In a further embodiment, the one or more additional vaccine is selected from the group consisting of: a Zika Virus vaccine; a Tick-Borne Encephalitis Virus vaccine; a Japanese Encephalitis Virus vaccine; a West Nile Virus vaccine; a Saint Louis Encephalitis Virus vaccine; an Omsk Haemorrhagic Fever Virus vaccine. Preferably, the one or more additional vaccine is selected from: TBEV; JEV; and Zika virus.

Flavivirus strains that may feature in a vaccine include those listed below.
1. TBEV Neudörfl (TBEV Eu, FSME and encepur-based strains) https://www.ncbi.nlm.nih.gov/protein/P14336.4
2. TBEV Sojifin (TBEV Far Eastern) https://www.ncbi.nlm.nih.gov/protein/130520
3. TBEV Fe-205 (TBEV EnceVir vaccine) https://www.ncbi.nlm.gov/nuccore/116109053
4. TBEV Senzhang (Changchun Institute of Biological Products, CIBP) https://www.ncbi.nlm.nih.gov/nuccore/AY182009.1
5. JEV Nakayama https://www.ncbi.nlm.nih.gov/nuccore/EF571853.1
6. JEV SA-14-14-2 (IXIARO strain+CD.JEVAX) https://www.ncbi.nlm.nih.gov/protein/AAA46248.1
7. ZIKV Polynesian strain 2013 http://www.ncbi.nlm.nih.gov/protein/631250743

Suitable TBE virus vaccines are listed below:

Encepur children: One dose (0.25 ml) contains: 0.75 micrograms inactivated TBE (tick-borne encephalitis) virus, strain K23*adsorbed on aluminum hydroxide (hydrogenated) (0.15-0.20 mg Al3+), Trometamol, Sucrose, sodium chloride, Water for injections.

Encepur adult: One dose (0.5 ml) contains 1.5 micrograms of inactivated TBE (tick-borne encephalitis) virus strain K23*adsorbed on aluminum hydroxide (hydrogenated) (0.3-0.4 mg Al 3+), trometamol, sucrose, sodium chloride, water for injections.

FSME Junior: Each dose (0.25 ml) of the suspension contains 1.2 micrograms inactivated TBE virus (strain neudorfl) grown in kycklingembryofibroblastcellkulturer (CEF cells) and adsorbed to aluminum hydroxide, hydrated (0.17 mg Al 3+). Human albumin, sodium chloride, disodium phosphate, potassium dihydrogen phosphate, sucrose, water for injections, hydrated aluminum hydroxide.

FSME Adult: Each dose (0.5 ml) of the suspension contains 2.4 micrograms inactivated TBE virus (strain neudorfl) grown in kycklingembryofibroblastcellkulturer (CEF cells) and adsorbed to aluminum hydroxide, hydrated (0.35 mg Al 3+). Human albumin, sodium chloride, disodium phosphate, potassium dihydrogen phosphate, sucrose, water for injections, hydrated aluminum hydroxide.

TBE Moscow: Each dose (0.5 ml) of the suspension contains 0.5-0.75 micrograms inactivated TBE virus (strain TBEV-FE Sojfin) grown in kycklingembryofibroblastcellculture (CEF cells) and adsorbed to aluminum hydroxide, hydrated (0.35 mg Al 3+). Human albumin, sodium chloride, disodium phosphate, potassium dihydrogen phosphate, sucrose, water for injections, hydrated aluminum hydroxide.

EnceVir: Each dose (0.5 ml) of the suspension contains 2.0-2.5 micrograms inactivated TBE virus (strain TBEV-Fe-205) grown in kycklingembryofibroblastcellculture (CEF cells) and adsorbed to aluminum hydroxide, hydrated (0.35 mg Al 3+). Human albumin, sodium chloride, disodium phosphate, potassium dihydrogen phosphate, sucrose, water for injections, hydrated aluminum hydroxide.

TBEV CIBP: Each dose (unknown amount) of the suspension contains inactivated TBE virus (strain TBEV-Fe Senzhang) grown in primary hamster kidney cells (PHKC), and adsorbed to aluminum hydroxide, hydrated (0.35 mg Al 3+). Human albumin, sodium chloride, disodium phosphate, potassium dihydrogen phosphate, water for injections, hydrated aluminum hydroxide.

A suitable JE virus vaccine is listed below:

IXIARO®:1 dose (0.5 ml) of IXIARO® contains Japanese encephalitis virus strain SA14-14-2 (inactivated) 1.2 6 AE3 corresponding to a power of ≤460 ng ED50. Phosphate buffered saline solution containing sodium chloride, potassium dihydrogen phosphate, disodium hydrogen phosphate, water for injections

It will be appreciated that vaccines currently in development may also be used. For example, a West Nile virus vaccine is currently being developed (see Brandler S, Tangy F. Vaccines in development against West Nile virus. Viruses 2013; 5(10): 2384-409), as is a Zika virus vaccine (see Vannice K S, Giersing B K, Kaslow D C, et al. Meeting Report: WHO consultation on considerations for regulatory expectations of Zika virus vaccines for use during an emergency. Vaccine 2016).

Suitable dosage regimes for selected vaccines against a Flavivirus are provided below.

| | | Conventional | Shortened |
|---|---|---|---|
| | Dose | schedule | schedule |
| IXIARO ® (adults) | | | |
| Dose 1 | 0.5 ml | Day 0 | Day 0 |
| | Intramuscular | | |
| Dose 2 | 0.5 ml | 28 days months | 7 days after |
| | Intramuscular | after first dose | first dose |
| Boosterdoses | 0.5 ml | 12-24 months | 12-24 months |
| | Intramuscular | after second dose | after second |
| | | | dose |
| IXIARO ® (24 | | | |
| months-3 yrs) | | | |
| Dose 1 | 0.25 ml | Day 0 | Day 0 |
| | Intramuscular | | |
| Dose 2 | 0.25 ml | 28 days months | 7 days after |
| | Intramuscular | after first dose | first dose |
| Boosterdoses | 0.25 ml | 12-24 months | 12-24 months |
| | Intramuscular | after second dose | after second |
| | | | dose |
| FSME-Vuxen | | | |
| immunization | | | |
| schedule-Adult | | | |
| (16 yrs and up) | | | |
| Dose 1 | 0.5 ml | Day 0 | Day 0 |
| | Intramuscular | | |
| Dose 2 | 0.5 ml | 1 to 3 months | 14 days after |
| | Intramuscular | after first dose | first dose |
| Dose 3 | 0.5 ml | 5 to 12 months | 5 to 12 months |
| | Intramuscular | after second dose | after second |
| | | | dose |
| Boosterdoses | 0.5 ml | Every 3-5 years | Every 3-5 years |
| | Intramuscular | after third dose | after third dose |
| FSME-Junior | | | |
| immunization | | | |
| schedule child | | | |
| (16 yrs and down) | | | |
| Dose 1 | 0.25 ml | Day 0 | Day 0 |
| | Intramuscular | | |
| Dose 2 | 0.25 ml | 1 to 3 months | 14 days after |
| | Intramuscular | after first dose | first dose |
| Dose 3 | 0.25 ml | 5 to 12 months | 5 to 12 months |
| | Intramuscular | after second dose | after second |
| | | | dose |
| Boosterdoses | 0.25 ml | Every 3-5 years | Every 3-5 years |
| | Intramuscular | after third dose | after third dose |
| Encepur | | | |
| immunization | | | |
| schedule-Adult | | | |
| (12 yrs and up) | | | |
| Dose 1 | 0.5 ml | Day 0 | Day 0 |
| | Intramuscular | | |
| Dose 2 | 0.5 ml | 1 to 3 months | 7 days after |
| | Intramuscular | after first dose | first dose |
| Dose 3 | 0.5 ml | 5 to 12 months | 21 days after |
| | Intramuscular | after second dose | second dose |
| Boosterdoses | 0.5 ml | Every 3 years | Every 3 years |
| | Intramuscular | after third dose | after third dose |
| Encepur Barn | | | |
| immunization | | | |
| schedule child | | | |
| (12 yrs and down) | | | |
| Dose 1 | 0.25 ml | Day 0 | Day 0 |
| | Intramuscular | | |
| Dose 2 | 0.25 ml | 1 to 3 months | 7 days after |
| | Intramuscular | after first dose | first dose |
| Dose 3 | 0.25 ml | 9 to 12 months | 21 days after |
| | Intramuscular | after second dose | second dose |
| Boosterdoses | 0.25 ml | Every 3 years | Every 3 years |
| | Intramuscular | after third dose | after third dose |
| TBE-Moscow | | | |
| immunization | | | |
| schedule | | | |
| Dose 1 | 0.5 ml | Day 0 | — |
| | Intramuscular | | |
| Dose 2 | 0.5 ml | 1 to 7 months | — |
| | Intramuscular | | |
| Dose 3 | 0.5 ml | 12 months | — |
| | Intramuscular | | |
| Boosterdoses | 0.5 ml | Every 3 years | — |
| | Intramuscular | | |
| EnceVir | | | |
| immunization | | | |
| schedule | | | |
| Dose 1 | 0.5 ml | Day 0 | Day 0 |
| | Intramuscular | | |
| Dose 2 | 0.5 ml | 5 to 7 months | 21-35 days |
| | Intramuscular | | |
| Dose 3 | 0.5 ml | 12 months | 42-70 days |
| | Intramuscular | | |
| Dose 4 | 0.5 ml | — | 6-12 months |
| | Intramuscular | | |
| Boosterdoses | 0.5 ml | Every 3 years | — |
| | Intramuscular | | |
| TBE- CIBP | | | |
| immunization | | | |
| schedule | | | |
| Dose 1 | Unknown | Day 0 | — |
| Dose 2 | Unknown | 14 days | — |
| Boosterdoses | Unknown | After 1 year | — |

It will be appreciated that one or more vaccine against the other Flavivirus typically further comprise a pharmaceutically acceptable carrier, diluent or adjuvant. Suitable carriers, diluents and adjuvants, routes of administration and ways of formulating the vaccine include those mentioned above in relation to the first aspect of the invention.

Further embodiments of the second and third aspects of the invention are described below.

Preferences for the other Flavivirus include those mentioned above in relation to the first aspect of the invention. Thus, the Flavivirus may be one or more Flavivirus selected from the group consisting of: Zika Virus; Tick-Borne Encephalitis Virus; Japanese Encephalitis Virus; West Nile Virus; Saint Louis Encephalitis Virus; Omsk Haemorrhagic Fever Virus. It is particularly preferred if the Flavivirus is one or more Flavivirus selected from the group consisting of: Zika Virus, Tick-Borne Encephalitis Virus and Japanese Encephalitis Virus.

In an embodiment, the Flavivirus is not Dengue Fever Virus.

In an embodiment, the one or more additional vaccine is a Tick-Borne Encephalitis Virus vaccine, and is for vaccinating the individual against infection by Tick-Borne Encephalitis.

In an embodiment, the one or more additional vaccine is a Japanese Encephalitis Virus vaccine and is for vaccinating the individual against infection by Japanese Encephalitis Virus.

In an embodiment, the one or more additional vaccine is a Zika Virus vaccine and is for vaccinating the individual against infection by Zika Virus.

In an embodiment, the one or more additional vaccine are a Tick-Borne Encephalitis Virus and a Japanese Encephalitis Virus vaccine, and are for vaccinating the individual against infection by Tick-Borne Encephalitis Virus and Japanese Encephalitis Virus.

In an embodiment, the one or more additional vaccine are a Tick-Borne Encephalitis Virus and a Zika Virus vaccine, and are for vaccinating the individual against infection by Tick-Borne Encephalitis Virus and Zika Virus.

In an embodiment, the one or more additional vaccine are a Japanese Encephalitis Virus and a Zika Virus vaccine, and are for vaccinating the individual against infection by Japanese Encephalitis Virus and Zika Virus.

In an embodiment, the one or more additional vaccine are a Japanese Encephalitis Virus vaccine, a Zika Virus vaccine, and a Tick-Borne Encephalitis Virus vaccine, and are for vaccinating the individual against infection by Japanese Encephalitis Virus, Zika Virus, and Tick-Borne Encephalitis Virus.

In an embodiment, the individual has not previously been infected with the Flavivirus and/or vaccinated against the Flavivirus. It is preferred if the individual has not previously been infected with the Flavivirus since it may be that life long immunity is created such that vaccination against that Flavivirus is unnecessary.

In an embodiment, the individual has not previously been infected with Yellow Fever Virus and/or vaccinated with the Yellow Fever Virus vaccine. It may be desirable if the individual has no previous immune memory to YFV since the YFV vaccine will then be the primary infection and generate a better immune response together with the one or more additional vaccine (eg TBEV or JEV).

In an embodiment, the individual has previously been infected with the Flavivirus and/or vaccinated against the Flavivirus.

In an embodiment, the individual has previously been infected with Yellow Fever Virus and/or vaccinated with the Yellow Fever Virus vaccine. The individual would still be expected to benefit from protection against the other Flavivirus but, without wishing to be bound by any theory, it is believed that the balance in the underlying mechanisms may be different. For example, compared to individuals that have not previously been infected with YFV and/or vaccinated with the YFV vaccine, the adjuvant effect of the YFV vaccine may be lower and the cross-reactivity caused by the YFV vaccine may be higher.

In an embodiment, the individual has previously been infected with the Flavivirus and/or vaccinated against the Flavivirus, but has not been previously infected with YFV and/or vaccinated against YFV.

In an embodiment, the individual has not previously been infected with the Flavivirus and/or vaccinated against the Flavivirus and has not been previously infected with YFV and/or vaccinated against YFV.

In an embodiment, the individual has previously been infected with the Flavivirus and/or vaccinated against the Flavivirus, and has previously been infected with YFV and/or vaccinated against YFV.

In an embodiment, the individual has not previously been infected with the Flavivirus and/or vaccinated against the Flavivirus and has previously been infected with YFV and/or vaccinated against YFV.

In a particularly preferred embodiment, the individual has not previously been infected with the Flavivirus and/or vaccinated against the Flavivirus and has not been previously infected with YFV and/or vaccinated against YFV.

It will be appreciated that vaccinating an individual against infection by the Flavivirus in the second and third aspects of the invention includes the same meaning as vaccinating an individual against infection by the Flavivirus in the first aspect of the invention, and so all of the preferences, limitations and definitions outlined in the first aspect of the invention equally apply to the second and third aspects of the invention.

For example, vaccinating the individual against infection by the Flavivirus in the context of the second and third aspect of the invention may prevent and/or reduce a subsequent infection by the Flavivirus. Similarly, vaccinating the individual against infection by the Flavivirus in the context of the second and third aspects of the invention may prevent and/or reduce one or more disorder and/or condition associated with infection by the Flavivirus. Methods for assessing such prevention and reduction are described above in relation to the first aspect of the invention.

Typically, the immunity against the Flavivirus that is provided in the context of the second and third aspects of the invention (eg the prevention and/or reduction in a subsequent infection by the Flavivirus, or the prevention and/or reduction in one or more disorders and/or conditions associated with infection by the Flavivirus) lasts for a period of up to 1 year, or 5 years or 10 years, as described above in relation to the first aspect of the invention.

Generally, the reduction in a subsequent infection by the Flavivirus and/or the reduction in the one or more disorder and/or condition associated with infection by the Flavivirus, that is afforded in the context of the second and third aspects of the invention, is a reduction by 100%, or by 90%, or by 80%, or by 70%, or by 60%, or by 50%.

Typically, the vaccination regime of the second and third aspects of the invention has a VE in providing immunity against another Flavivirus of at least 50%, such as at least 55%, 60%, 65%, 70%, 75%, 80%, 85%, 90% or 95%. It is preferred if the vaccination regime has at least 85% VE. Measuring VE may be done by any of the methods described above in relation to the first aspect of the invention.

As described above in relation to the first aspect of the invention, vaccinating the individual against infection by the Flavivirus in the context of the second or third aspect of the invention may generate and/or increase immunity in the individual against the Flavivirus. The immunity may comprise cellular immunity and/or adaptive immunity in the individual against the Flavivirus, and preferences for such immunity include those described above in relation to the first aspect of the invention.

Without wishing to be bound by any theory, the inventors believe that administering both a YFV vaccine and one or more vaccine against a Flavivirus to an individual in accordance with the second and third aspects of the invention, acts synergistically to provide protective immunity against the Flavivirus. For example, as described in more detail in the Examples, the inventors have shown that antibody-producing plasmablasts increase dramatically if given two vaccines instead of one. This means that more antibodies to the infections are produced than if given one vaccine. Further, TBE-T cells arise if given two vaccines, whereas this is not achieved if given several single-TBE doses, thereby indicating synergy in B and T cell compartments. The inventors believe that such synergy allows for lower and/or fewer doses of the respective vaccines, both of which can increase compliance in the patient population. Lower and/or fewer doses are also more cost effective. The inventors also believe that such synergy may avoid the need for adjuvant Flavivirus vaccines. For example, alum-adjuvant in the TBE/JE vaccines may not be needed if YFV+TBE and YFV+JE vaccines can be used.

The inventors believe that the YFV vaccine not only increases the immunity afforded by other Flavivirus vaccines, but is expected to act as a general adjuvant for other vaccines. Thus, a fourth aspect of the invention provides the use of a Yellow Fever Virus vaccine as an adjuvant.

Preferences for the YFV vaccine include those described above in relation to the first aspect of the invention. For example, it is preferred if the YFV is a live attenuated vaccine such as Stamaril®.

By adjuvant we include the meaning that the YFV vaccine potentiates the immune response to an antigen. In other words, when the antigen and the YFV vaccine are administered to an individual the immune response to the antigen is greater than the immune response to the antigen had the YFV vaccine not been administered together with it. For example, the immune response may be at least 1 fold, 2 fold, 3 fold, 4 fold, 5 fold or 10 fold greater, or even at least 20 fold greater such as 50 fold greater or 100 fold greater. Typically, the immune response may be at least 1-3 fold greater, but it will be appreciated that the presence of the adjuvant may be the difference between a detectable immune response and none at all.

In an embodiment, the YFV vaccine is used as an adjuvant for a vaccine such as an inactivated vaccine. However, it will be appreciated that the YFV vaccine may be used as an adjuvant for other forms of vaccine such as DNA and protein based vaccines.

The inactivated vaccine may be a viral vaccine such as any of Hepatitis A Vaccine, Influenza Vaccine (shot), Rabies Vaccine, Japanese Encephalitis Vaccine.

The inactivated vaccine may be a bacterial vaccine such as any of Inactivated Typhoid Vaccine, Inactivated Cholera Vaccine, Plague Vaccine, Diptheria, Tetanus and Pertussis Vaccine(s).

The inactivated vaccine may be a subunit vaccine such as Hepatitis B or HPV Vaccine.

It will be appreciated that this aspect of the invention therefore includes a method of potentiating the immune response to an antigen (eg a vaccine such as an inactivated vaccine), the method comprising administering a Yellow Fever Virus vaccine to an individual who is administered the antigen. The antigen may be administered substantially simultaneously to the YFV vaccine, but it is appreciated that they may be administered sequentially. Preferably, the YFV and antigen are administered simultaneously.

As used herein in relation to all aspects of the invention, the term “individual” is preferably a mammalian individual. Preferably, the mammal is a human, although it will be appreciated that the individual may be a non-human mammal, such as any of a horse, dog, pig, cow, sheep, rat, mouse, guinea pig or a primate.

It will be appreciated that some Flaviviruses are prevalent in the pediatric population, and so in one embodiment, the individual is a child (ie under 18 years of age). The inventors believe that the vaccine regimes of the invention (ie in accordance with the first, second and third aspects of the invention) will be useful in preventing later infections and it may be desirable to vaccinate individuals at an early age (eg from 6 months such as 1 year, 2 years, 3 years, 4 years or 5 years).

A fifth aspect of the invention provides a vaccine composition comprising a Yellow Fever Virus vaccine, and one or more additional vaccine against a Flavivirus, and a pharmaceutically-acceptable excipient or diluent.

Preferences for the YFV vaccine and one or more additional vaccine against a Flavivirus include those mentioned above in relation to the first, second and third aspects of the invention.

A sixth aspect of the invention provides a kit comprising: a Yellow Fever Virus vaccine and one or more vaccine against a Flavivirus. Again, preferences for the YFV vaccine and one or more additional vaccine against a Flavivirus include those mentioned above in relation to the first, second and third aspects of the invention.

The vaccine composition of the fifth aspect of the invention and the kit of the sixth aspect of the invention may include at least 2, 3, 4, 5, 6, 7, 8, 9, 10 or more vaccines against a Flavivirus.

When two or more vaccines against a Flavivirus are used, it will be appreciated that the two or more vaccines against a Flavivirus against a Flavivirus may be two or more vaccines against the same Flavivirus, or against different Flaviviruses. Thus, the two or more vaccines against a Flavivirus may comprise two or more vaccines against different Flaviviruses respectively, eg 3 or more different Flaviviruses, 4 or more different Flaviviruses, and so on.

In an embodiment, the vaccine composition of the fifth aspect of the invention or the kit of the sixth aspect of the invention comprises a TBE virus vaccine.

In an embodiment, the vaccine composition of the fifth aspect of the invention or the kit of the sixth aspect of the invention comprises a JEV vaccine.

In an embodiment, the vaccine composition of the fifth aspect of the invention or the kit of the sixth aspect of the invention comprises a Zika virus vaccine.

In an embodiment, the vaccine composition of the fifth aspect of the invention or the kit of the sixth aspect of the invention comprises a TBE virus vaccine and a JEV vaccine.

In an embodiment, the vaccine composition of the fifth aspect of the invention or the kit of the sixth aspect of the invention comprises a TBE virus vaccine and a Zika virus vaccine.

In an embodiment, the vaccine composition of the fifth aspect of the invention or the kit of the sixth aspect of the invention comprises a Zika virus vaccine and a JEV vaccine.

In an embodiment, the vaccine composition of the fifth aspect of the invention or the kit of the sixth aspect of the invention comprises a TBE virus vaccine and a JEV vaccine and a Zika virus vaccine.

A seventh aspect of the invention provides a method of producing an immunological serum, the method comprising:
(a) vaccinating an individual with the vaccine composition of the second or third aspect of the invention; and
(b) obtaining immunological serum from the individual.

Obtaining immunological serum can be done by any suitable method in the art, including that described in McKinney et al (J Immunol Methods 1987: 96(2): 271-8).

It will be appreciated that the invention includes an immunological serum obtainable by the method of the seventh aspect of the invention.

It will be appreciated that the immunological serum so produced may be used to provide passive immunity in a recipient individual, for example in an individual with an immunodeficiency or with an immunosuppression, or in an individual which requires rapid neutralisation of virus particles. Thus, the immunological serum may be used as part of intravenous immunoglobulin treatment (Weissbach F H, Hirsch H H. Comparison of Two Commercial Tick-Borne Encephalitis Virus IgG Enzyme-Linked Immunosorbent Assays. Clin Vaccine Immunol 2015; 22(7): 754-60 and Jolles et al, Clin Exp Immunol 2005; 142(1): 1-11)).

It will be appreciated that the invention therefore includes a method of providing passive immunity to an individual, the method comprising administering to the individual immunological serum obtained from an individual that has been vaccinated with the vaccine composition of the second or third aspect of the invention. It is appreciated that the immunological serum may be obtained from a population of individuals that have been vaccinated with the vaccine composition of the second or third aspects of the invention.

For example, intravenous immunoglobulin (IVIG) may be a blood product prepared from the serum of between 1000 and 15 000 donors per batch. It is the treatment of choice for patients with antibody deficiencies. For this indication, IVIG may be used at ‘replacement dose’ of 200-400 mg/kg body weight, given approximately 3-weekly. In contrast, ‘high dose’ IVIG (hdIVIG), given most frequently at 2 g/kg/month, is used as an ‘immunomodulatory’ agent in an increasing number of immune and inflammatory disorders. Initial use of hdIVIG was for immune thrombocytopenic purpura (ITP) in children. It will be understood that by passive immunity we include the administration of both IVIG and hdIVIG.

The invention includes a vaccine composition for use, or a use, or a method, substantially as claimed herein with reference to the accompanying claims, description, examples and figures.

The invention includes a vaccine composition substantially as claimed herein with reference to the accompanying claims, description, examples and figures.

The invention includes a kit substantially as claimed herein with reference to the accompanying claims, description, examples and figures.

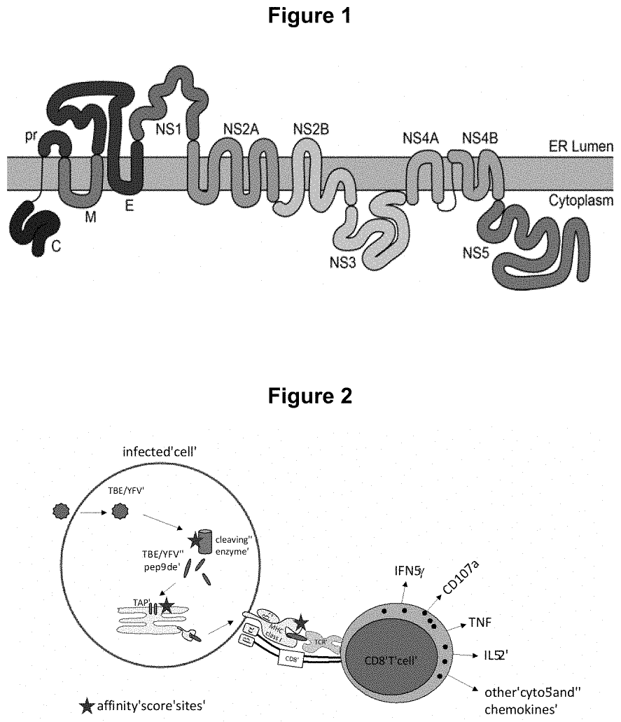

FIG. 1. Flavivirus RNA encodes for 10 proteins.

FIG. 2. Schematic figure of the NetCTL prediction score sites.

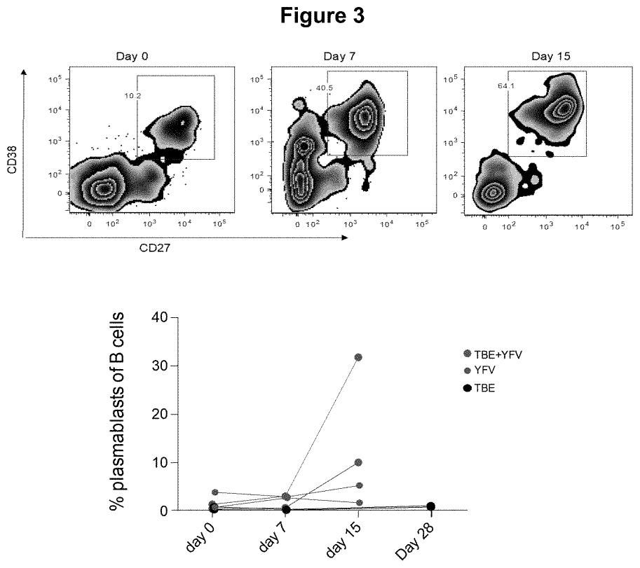

FIG. 3. Appearance of short-lived antibody-secreting plasmablasts at day 0, 7 and 15 or 28 (28 after TBE vaccine) after vaccinations. (A) Flow plot showing the plasmablasts in one alpha donor before and at day 7 and 15 after vaccination. (B) Plots showing the plasmablasts after administration with either YFV alone (blue), TBE vaccine alone (black), and YFV and TBE vaccines administered at the same time (red). Plots show plasmablasts in percent of total B cells.

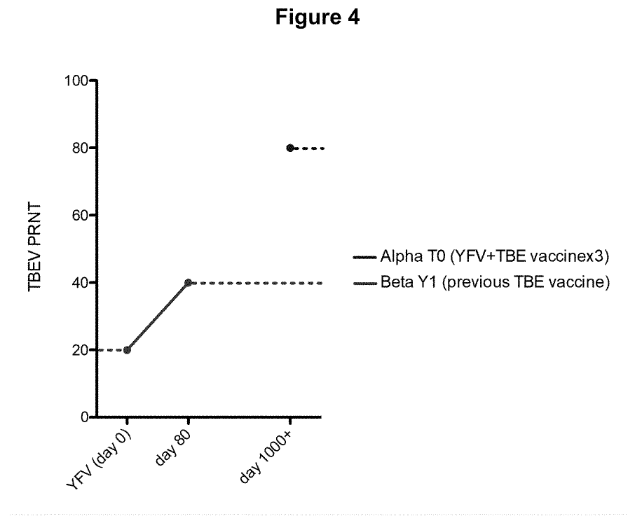

FIG. 4. Levels of TBEV neutralizing antibodies after yellow fever vaccination. TBEV neutralizing antibodies in sera from three donors vaccinated with YFV vaccine. Alpha T0 received concomitant YFV and TBE prime vaccines, and thereafter two additional doses of TBE vaccine. Sampling time point was three year after primary immunizations, and levels of neutralizing antibodies measured to 80%. Beta Y1 received several TBE vaccines previously, with the last booster 4 months prior YFV vaccine and levels of neutralizing antibodies increase from 20% (before YFV vaccine) to 40% (80 days after YFV vaccine). Beta Y2 is TBE vaccine negative, and received YFV vaccine and no TBEV neutralizing antibodies were detected (data not shown).

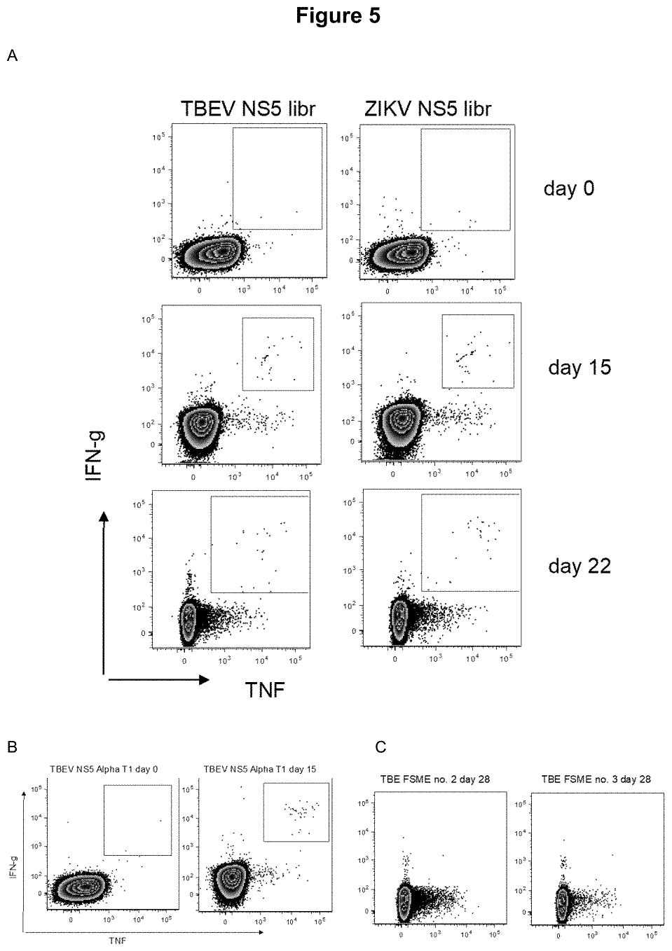

FIG. 5. Cross reactive CD4 T cells against TBEV and ZIKV arise after concomitant administration of YFV and TBE vaccines. (A) PBMC from before and at days 15 and 22 after concomitant vaccinations were activated with ZIKV or TBEV overlapping NS5 libraries in one donor (alpha T2). (B) PBMC from before and at day 15 after vaccinations were activated with TBEV overlapping NS5 libraries in one donor (alpha T1). (C) PBMCs activated with TBEV overlapping NS5 libraries in one donor from day 28 after 2:nd and 3:rd dose of FMSE.

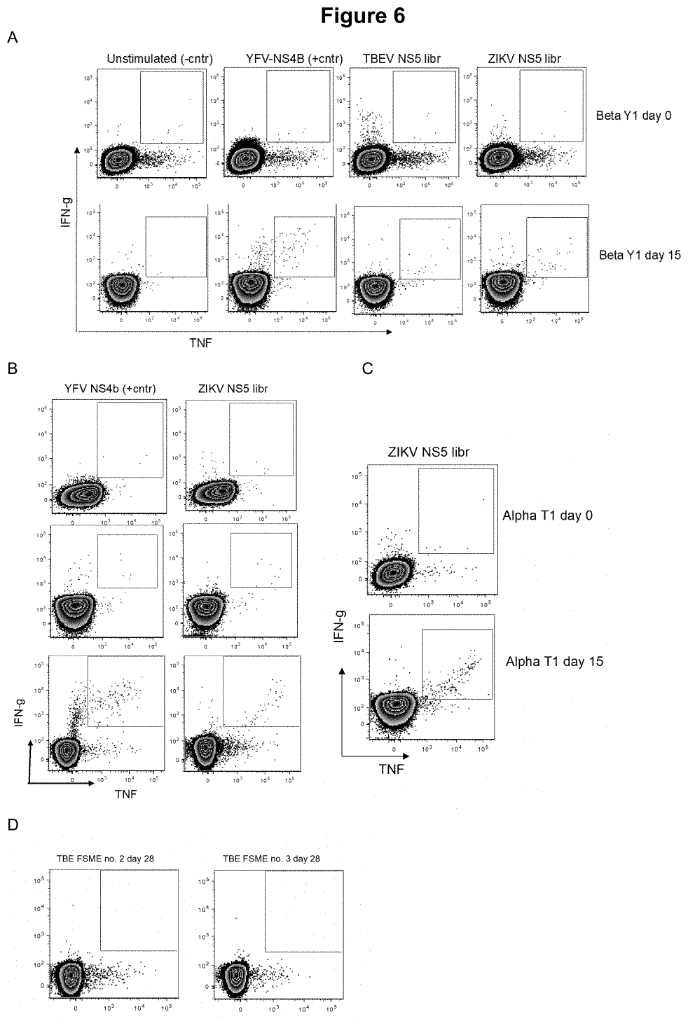

FIG. 6. Cross-reactive CD8 T cells against TBEV and ZIKV arise after administration with YFV and TBE vaccines. (A) Plots show IFN-γ and TNF double positive CD8 T cells after activation with either nothing (−cntr), YFV-NS4B covering library (+cntr), TBEV NS5 covering peptide library or ZIKV NS5 covering library in one donor vaccinated with YFV only (beta Y1). (B) Plots show IFN-γ and TNF double positive CD8 T cells at day 0 (top), 15 (middle) and 22 (bottom) after vaccination. Cells were activated with either YFV-NS4B covering library (+cntr), or ZIKV NS5 covering peptide library in one donor (alpha T2) receiving concomitant YFV and TBE vaccinations. (C) Plots show IFN-γ and TNF double positive CD8 T cells over time after activation with ZIKV NS5 covering peptide library in one donor (alpha T1) receiving concomitant YFV and TBE vaccinations. (D) Plots show PBMC from a TBE single vaccine, 28 days after administration after second and third TBE booster dose, activated with TBEV NS5 peptide library.

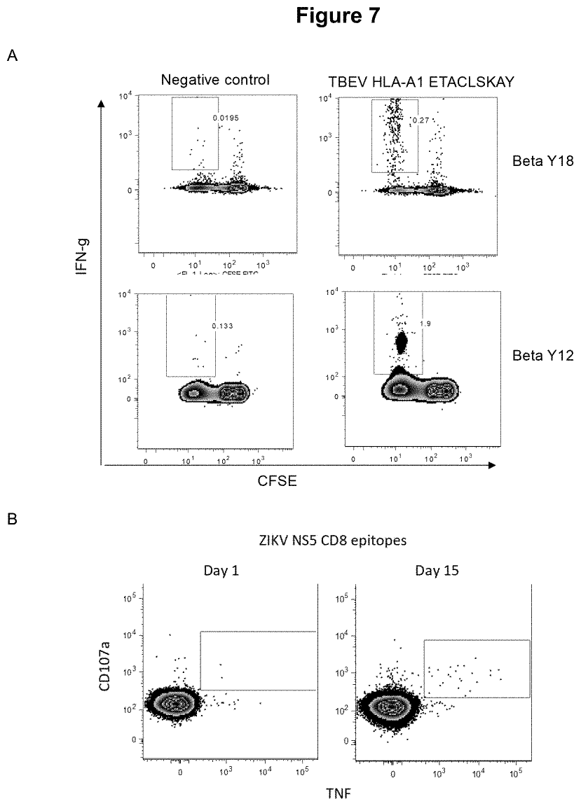

FIG. 7. Cross reactive T cells against TBEV and ZIKV arise after vaccination with YFV vaccine alone. (A) PBMC from two YFV vaccinated donors (HLA-A1 positive) were expanded for 6 days with either nothing (negative control, left panel), or predicted TBEV peptide ETACLSKAY (SEQ ID NO: 1) (right panel), 15 days after vaccination with YFV. Specific cells are measured as proliferating (CFSE low) and IFN-γ positive. (B) T cells 1 day and 15 days after vaccination were activated for 6h with predicted ZIKV NS5 CD8 epitopes. Specific cells are measured by the simultaneous expression of CD107a (degranulation) and TNF.

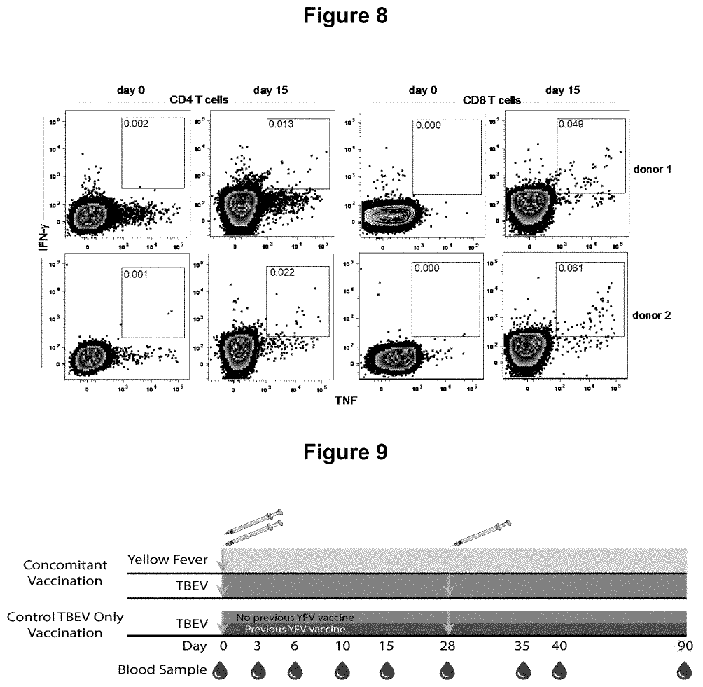

FIG. 8. Zika virus-specific T cells arise after yellow fever vaccination. PBMCs from two individuals (donor 1 and donor 2), activated with a Zika virus-specific NS5 library, before (day 0) and at day 15 following YFV vaccination. CD4 and CD8 T cells responding to the Zika-NS5 library are identified by production of IFN-γ and TNF. Numbers in figure indicate percentage of CD4 T cells and CD8 T cells.

FIG. 9. Vaccination and blood draw schedule for concomitant YFV and TBEV. A booster TBEV vaccine will be given at day 28 with blood draw the same day.

FIG. 10. Viral strains derived from Asibi strains.

FIG. 11. JEV NS5 stimulations of CD8 T cells (12h) on frozen PBMCs from a healthy donor receiving YVF vaccine (donor By4), before (day 0) and at 15 days after vaccination. Production of TNF increases from 0.03% to 0.3% which is a 10-fold increase in response to the JEV NS5 peptide library.

### EXAMPLE 1: YFV VACCINE INDUCES/ENHANCES IMMUNE RESPONSE AGAINST OTHER FLAVIVIRUSES

Flaviviruses belong to the family of Flaviviridae and comprise over 70 viruses that cause severe diseases. These viruses are responsible for hundreds of thousands of deaths annually and additional significant morbidity. There are currently commercially available vaccines against three flaviviruses; YFV, JEV and TBEV (Table 1). Of these vaccines, the live attenuated YFV vaccine is one of the most effective and uses vaccines on earth.

TABLE 1
Overview of flaviviruses, endemic area and available vaccines.

| Virus | Vector | Endemic area | Vaccine available |
|---|---|---|---|
| JEV | Mosquito (Culex) | Asia | IXIARO ®-Inactivated |
| ZIKV | Mosquito (Aedes) | Africa, | None available |
| | | Latin America | |
| YFV | | | Stamaril ®/YF-VAX ®- |
| | | | Live attenuated |
| TBE | Tick (Ixodes) | Eurasia | FSME-IMMUN and Encepur- |
| | | | Inactivated |

This Example investigates the cross-reactivity of the YFV vaccine to other Flaviviruses, and in particular to TBEV and 21 KV.

### Methods

### Peptide Libraries

Peptide libraries are used to display multiple, linear peptide fragments in parallel to deduce specific epitopes. We have designed peptide libraries covering TBEV and ZIKV NS5 proteins, designed to activate both CD4 and CD8 T cells, with overlapping peptide segments of the entire antigen. Binding is assessed by flow cytometry and this method has been especially useful for developing vaccines.

### Prediction of CD8 T Cell Epitopes

To identify virus-specific CD8 T cells with flow cytometry, T cell epitopes in the virus have to be identified if they are not already known. Epitope identification requires a systematic screening of the antigen, which can be difficult when the antigen has multiple conformations or binding domains. Another method to identify T cell epitopes to a specific pathogen is to utilize online search engines, which consider HLA-type and possible peptide presentation by the MHC molecule on the cell surface. The traditional whole genome library have the advantage of being HLA-unbiased while predicative algorithms represent a more high throughput technology. We have generated ZIKV and TBEV CD8 pools of predicted epitopes in ZIKV (Table 2) and TBEV (Table 3) NS5 proteins to the most common HLA-types (HLA-A1, A2, A3, B7 and B8). We used the NetCTL search engine to predict the epitopes, which integrates prediction of peptide-MHC class I binding, proteasomal C terminal cleavage and TAP transport efficiency (FIG. 2).

TABLE 2
Predicted ZIKV epitopes.

| | | | HLA | NetCTL |
|---|---|---|---|---|
| Nr | ZIKV | SEQ ID NO: | Supertype | Score |
| 1 | ETACLAKSY | 1 | A1 | 1.7742 |
| 2 | RTWAYHGSY | 2 | A1 | 1.6323 |
| 3 | ALNTFTNLV | 3 | A2 | 1.2792 |
| 4 | YMWLGARFL | 4 | A2 | 1.1720 |
| 5 | RTTWSIHGK | 5 | A3 | 1.3336 |
| 6 | CVYNMMGKR | 6 | A3 | 1.2696 |
| 7 | GLVRVPLSR | 7 | A3 | 1.1146 |
| 8 | YTYQNKVVK | 8 | A3 | 0.9942 |
| 9 | NMMGKREKK | 9 | A3 | 0.8719 |
| 10 | GLGLQRLGY | 10 | A3 | 0.8095 |
| 11 | VPCRHQDEL | 11 | B7 | 0.9698 |
| 12 | GLQRLGYVL | 12 | B8, A2 | 1.1986 |
| 13 | WLGARFLEF | 13 | B8 | 1.0892 |
| 14 | LLYFHRRDL | 14 | B8, A2 | 1.0109 |
| 15 | SGQVVTYAL | 15 | B8 | 1.0040 |

TABLE 3
Predicted TBEV epitopes.

| | | | HLA | NetCTL |
|---|---|---|---|---|
| Nr | TBEV | SEQ ID NO: | Supertype | score |
| 1 | STLNGGLFY | 16 | A1 | 2.9180 |
| 2 | ETACLSKAY | 17 | A1 | 2.0304 |
| 3 | GVEGISLNY | 18 | A1 | 1.9546 |
| 4 | VMEWRDVPY | 19 | A1 | 1.2679 |
| 5 | VLAPYRPEV | 20 | A2 | 1.2973 |
| 6 | YMWLGSRFL | 21 | A2, B8 | 1.1707 |
| 7 | MLVSGDDCV | 22 | A2 | 0.8937 |
| 8 | YALNTLTNI | 23 | A2 | 0.8088 |
| 9 | TLTNIKVQL | 24 | A2 | 0.7921 |
| 10 | CVYNMMGKR | 25 | A3 | 1.2472 |
| 11 | KLGEFGVAK | 26 | A3 | 1.2261 |
| 12 | AKVKSNAAL | 27 | B7 | 0.8632 |
| 13 | VVTYALNTL | 28 | B7 | 0.8505 |
| 14 | IAKVKSNAA | 29 | B8 | 1.4929 |
| 15 | SGQVVTYAL | 30 | B8 | 1.0018 |

### Study Design and Subjects

Peripheral blood and sera was collected before and at days 7 and 15 from four donors vaccinated with a primary dose of YFV vaccine (Beta Y donors), and before and at days 7, 15 and 22 from two donors that received concomitant vaccination with primary doses of YFV and TBE (alpha T donors). As a negative control group, PBMCs were collected at day 28 from the second and third dose from individuals vaccinated with TBE (FSME) only. PBMC were isolated from EDTA tubes (BD Biosciences, San Jose, Calif.). PBMC were either stained fresh or cryopreserved in 90% FCS and 10% DMSO for later analysis.

### Antibodies for Flow Cytometry

Immune responses were assessed using multi-color flow cytometry, and the monoclonal antibodies (mAbs) used were: anti-CD107a FITC, anti-CD4 brilliant ultraviolet 395, anti-CD19 Brilliant ultra violet 395, anti-CD4 brilliant ultraviolet 737, anti-HLA-DR APC, anti-Ki67 Alexa Fluor 700, anti-MIP-1β Alexa Fluor 700, anti-CD14 BD horizon V500, anti-CD19 BD horizon V500 and anti-TNF PE-CF594 and were all from BD Biosciences (San Jose, Calif.). Anti-CD45RA APC-Cy7, anti-IFN-γ Brilliant Violet 421, anti-CD27 Brilliant Violet 650, anti-CD38 brilliant violet 785, anti-IgG PE, anti IgD PE-Cy7 were all from Biolegend (San Diego, Calif.). Anti-CD8 Qdot 605 and Aqua Live/Dead were all from Invitrogen (Carlsbad, Calif.). Anti-CD3 PE-Cy5 and anti-CD56 ECD were from Beckman Coulter (Brea, Calif.).

### Flow Cytometry

For phenotypic analysis of cells, PBMCs were incubated for 30 minutes 4° C. in the dark, with surface mAbs, followed by washing with PBS. For the CD107a staining, the CD107a antibody was present during the 6 hours stimulation, and then additional CD107a antibody was added together with the surface mAbs for 30 minutes incubation at 4° C. in the dark. Cells were fixed and permeabilized with fix/perm (eBioscience) for 30 minutes at 4° C. in the dark. Cells were then washed and stained with intracellular mAbs. Samples were acquired on a BD LSRFortessa instrument (BD Biosciences) and analyzed using FlowJo software version 9.4 (Tree Star, Ashland, Oreg.). B cell plasmablasts were identified as lymphocytes (extended gate), single cells, live cells and CD14/CD123 negative, CD3 and CD4 negative, CD20 negative and CD19 positive, CD27 and CD38 double positive. T cells were identified as lymphocytes, singlets, dump negative (CD19, CD14 and aqua dead cell marker) and positive for CD3, CD4 or CD8 positive.

### In Vitro Functional Assays

PBMCs were rested in RPMI 1640 medium containing 10% FCS, 2 mM L-glutamine, 1% penicillin and streptomycin (Invitrogen) overnight at 37° C. Cells were stimulated with 10 μg peptides for 12 or 6 hours in 96-well round bottom plates in the presence of Brefeldin A (Sigma-Aldrich, St. Louis, Mo.), monensin (BD Biosciences) and purified anti-CD28/CD49d (1 μl/ml) (BD Bioscienses). Staining, flow cytometry and analyses were performed as described above.

### Plaque Reduction Neutralization Test (PRNT)

Sera from vaccinated individuals were prepared according to standard clinical diagnostic protocol at Swedish Institute for infectious disease control²². Sera, including positive and negative controls, were inactivated and diluted two-fold in Hanks' basal salt solution with 2% inactivated FCS, 2% 1 M HEPES. Equal amounts of serum dilutions and virus at approximately 50 PFU: 100m l were mixed and tubes were incubated at 37° C. and 5% CO2 for 1 h. Following incubation, 200 μl of the serum-virus mixture was added to the plates with Vero cell monolayers. After incubation at 37° C. and 5% CO2 for 1 h, the wells were overlaid with 2 ml mixture of one part 1% agarose and one part 2 basal Eagle's medium supplemented with 8% FCS, 2% 1 M HEPES. The plates were incubated at 37° C. and 5% CO2 for 6 days, when a second overlay containing 3.3% neutral red was added at 2 ml/well. Plates were returned to the incubator and plaques were enumerated the following day. The test was accepted if the virus dose was in the range 30-70 PFU. Neutralizing antibody titres were calculated as the reciprocal of the serum dilution that gave an 80% reduction of the number of plaques, as compared to the virus control.

### Results

### High Cross-Homology in Flavivirus NS5 Protein

NS5 is a multifunctional, conserved protein in flaviviruses and constitutes the viral polymerase. We compared the sequences the NS5 protein in YFV, TBEV JEV and ZIKV and found that, YFV NS5 protein has over 60% homology with the NS5 proteins of TBEV, JEV and ZIKV (Table 4).

TABLE 4
Results after protein BLAST of flavivirus protein sequences.

| | Complete protein | NS5 protein |
|---|---|---|
| Virus and strain (vaccine | homology to YFV- | homology to YFV- |
| name) | 17D204 (%) | 17D204 (%) |
| YFV 17D204 (Stamaril ®) | 100 | 100 |
| JEV-SA14-14-2 (IXIARO ®) | 45 | 63 |
| TBEV Neudeurfl (FSME ®) | 43 | 61 |
| ZIKV-polynesian isolate | 46 | 64 |
| West Nile virus-human isolate | 46 | 63 |
| St. Louis encephalitis | 47 | 65 |
| virus-2015 | | |
| Omsk hemorrhagic fever virus | 43 | 61 |
| Murray valley encephalitis | No virus sequence | — |
| virus | available | |

| | Complete protein | NS3 protein |
|---|---|---|
| | homology to YFV | homology to YFV |
| Virus | 17D204 (%) | 17D204 (%) |
| YFV 17D204 | 100 | 100 |
| JE-SA14-14-2 | 45 | 55 |
| TBEV Neudeurfl | 43 | 48 |
| ZIKV-polynesian | 46 | 56 |
| WNV-human isolate | 46 | 57 |
| SLEV-2015 | 47 | 58 |
| OHFV | 43 | 47 |
| MVEV | ND | ND |

We isolated blood from individuals vaccinated with either YFV alone, or individuals that received concomitant vaccinations with YFV and TBE vaccines.

Concomitant YFV and TBE Vaccination Generates a Higher Amount of Antibody Producing B Cells than the YFV Vaccine Alone

Immune responses after vaccination can be measured in multiple ways. One of the earliest responding immune cells in infection and vaccination are B cells. Germinal centers are transiently developed after antigen/vaccine exposure and are critical sites for selecting and differentiating B cells to become short-lived antibody-producing plasmablasts and memory B cells. Plasmablast responses have been shown to be a predictive measure of antibody levels induced by vaccination and can therefore aid in early evaluation of the efficacy of a vaccine²³. Plasmablasts can be detected by flow cytometry in the acute-stage of infection²⁴, however it is a bit more challenging to detect after primary vaccination, due to low amounts generated. Several booster doses are usually required to detect them after vaccination.

To assess the appearance of plasmablasts after vaccinations, we stained freshly isolated PBMCs before and at day 7 and 15 after administration with either YFV alone (Beta Y donors), or after concomitant administration of YFV and TBE (alpha T donors) (FIGS. 3A and B). Plasmablasts are visible in both cohorts at day 7 after vaccination, however the alpha donors have a peak with numbers as high as 10-30% of total B cells at day 15 (FIGS. 3A and B). These are levels measurable with acute dengue infection²⁴.

To measure the neutralizing effect of the antibodies generated, we performed a plaque reduction neutralization test (PRNT) for TBEV22 on three donors vaccinated with the YFV vaccine. One donor was previously negative for both YFV and TBE vaccines (Beta Y2), one donor received several TBE vaccines previously, with the last booster 4 months prior YFV vaccine (Beta Y1), and one donor received concomitant YFV and TBE prime vaccines, and had thereafter followed a full TBE vaccine program (two additional doses of TBE vaccine, with the last booster dose four months prior sampling time point (Alpha T0).

Beta Y1 showed a 100% increase (from 20-40% neutralization) before and after administration of YFV vaccine (FIG. 4). Furthermore, Alpha T0 measured highest levels of neutralizing antibodies against TBEV (80% neutralization). Together this indicates that 1; YFV vaccine does not generate cross neutralizing antibodies against TBEV (measured by this assay); 2; YFV vaccine boost already existing TBEV Nab and 3; a high degree of TBEV Nab is generated if prime YFV and TBE vaccines are given concomitantly.

### YFV Vaccine Generates Protective Cross-Reactive CD4 T Cells to TBEV and ZIKV

CD4 T cells responding to antigens have critical functions in activating and regulating immune responses via the production and release of various cytokines and play an important role in the protective immunity against viruses, primed by infection or by vaccination. CD4 T cells can promote contact of CD8 T cells with DCs in secondary lymphoid tissue^{25,26}, as well as recruit lymphoid cells into draining lymph nodes 27, and recruit innate or antigen-specific effectors to the site of viral replication^{28,29}. CD4 T cells can be divided into distinct subsets depending on their cytokine profile. In a simplified view, Th1 subsets activate cellular immune responses via the production of IFN-γ, TNF and IL-2, and Th2 subsets mainly produce cytokines supporting B cell activation 30.

To study whether YFV and TBEV specific, as well ZIKV cross reactive CD4 T cells arise after vaccination, we cultured PBMC with TBEV or ZIKV NS5 covering libraries from day 0 and 15 (FIG. 5) from donors vaccinated with YFV and TBE. In donor alpha T2, specific cells against both TBEV and ZIKV NS5 libraries could be detected. ZIKV specific cells also appeared at day 15 in the double vaccinated alpha T1 donor (FIG. 5B). However, TBE vaccine alone appear not to induce CD4 specific T cells after the second of third dose of TBE vaccine (FIG. 5C).

YFV Vaccine Enhances Production of TBEV Specific T Cells after Concomitant Vaccination with TBEV and Generates Cross-Reactive T Cells Against ZIKV

CD8 T cells have a spectrum of functions to control viral infections. Here, we assessed cytokine expression (IFN-γ and TNF) and degranulation (CD107a) of after activation with TBE/ZIKV NS5 libraries (FIG. 6) or with predicted ZIKV/TBEV CD8 T cell pools (FIG. 6) from before (day 0) and at days 15 and 22 after vaccinations. YFV alone generates a small amount of cross-reactive CD8 T cells against both TBEV NS5 and ZIKV NS5 at day 15 in donor beta Y1 (FIG. 6A). ZIKV specific CD8 T cells are absent before, but detectable at 15 days after concomitant administration with YFV and TBEV vaccines (FIGS. 6B and C). These cells are completely absent after multiple second and third dose of the TBE vaccine (FIG. 6D), demonstrating that TBEV vaccine alone may not generate specific CD8 T cells. Together, this indicates that TBEV CD8 T cells are generated after one single dose of the TBE vaccine if given simultaneously as the YFV vaccine.

To assess the existence of CD8 T cell responses against single TBEV peptide sequences (Table 3), we CFSE-labeled PBMCs from 2 HLA-A1 positive donors recently vaccinated with YFV, and cells were cultured with the corresponding synthetic peptides for 6 d. By the end of day 6, these cultures were re-stimulated for 12 h in the presence of peptide, and responses were determined by intracellular staining for IFN-γ (FIG. 7A). Responding cells were identified as double positive for CFSE dilution, indicative of proliferation during culture with peptide, and IFN-γ production. One peptide, predicted to be presented in HLA-A1 positive donors induced responses in both donors, with up to 1.9% IFN-γ positive cells. Next, we activated T cells with the predicted CD8 ZIKV pool (Table 2) in one donor previously activated with the YFV vaccine from before and at 15 days after vaccination. ZIKV specific T cells, producing CD107a and TNF arise at day 15 after YFV administration.

### Conclusion

With these data, we show that antibody-producing plasmablasts arise and peak at day 15 after administration of YFV and YFV together with TBE vaccine. The levels of plasmablasts were higher in individuals receiving concomitant vaccinations as compared to individuals receiving the YFV alone (FIG. 3). This may indicate that there is a synergy-adjuvant effect generated when receiving concomitant vaccinations of TBEV and YFV.

The YFV alone generate small but detectable protective cross-reactive CD8 T cells against ZIKV, JEV and TBEV (FIGS. 6 and 7). However in the individuals receiving concomitant vaccinations with YFV and TBE, a better CD4 T cell response against TBEV NS5 peptides was detected, as compared to individuals that receive the TBE vaccine alone (FIG. 5). Individuals receiving concomitant vaccinations with YFV and TBE show a strong CD8 T cell response against ZIKV NS5 library (FIG. 5), indicating cross-species protective effects of flavivirus vaccines.

### Discussion

We have for several years established and studied a cohort of healthy volunteers that were vaccinated with the YFV with a focus on how NK and T cell responses evolve over time. Vaccines take years to develop, which can be detrimental to global health with emerging pathogens. If we had a better understanding of how flavivirus vaccines work and the responses they induce, we could better design vaccines towards emerging viruses like ZIKV and/or improve the TBEV or JEV vaccines or immunization schedules. There is no commercial vaccine for ZIKV yet, but cross-reactivity in immunity gained from other flavivirus vaccinations could potentially aid in protecting those in high-risk areas from infection. Furthermore, if future ZIKV vaccines prove not to be entirely effective, co-vaccination with the YFV vaccine may yield improved vaccination.

The TBE and JE vaccines are relatively weak that require multiple booster doses, and with the number of vaccine failures reported in recent years, there is great need for improvement. If simultaneous immunizations lead to more robust immune responses, better protection against the infection could be acquired and less vaccine failures would occur. The results would also lead to more cost effective vaccination regimens as fewer boosters are required, and those vaccinated would sustain sufficient protection for a longer period. The vaccinations could be made more economically available for people living in TBEV or JEV endemic areas, ultimately reducing the number of infections and thereby fewer deaths or life long complications associated with the diseases.

Investigating the cross-reactivity of vaccines to related pathogens is important for understanding how immune responses to vaccines develop, and the mechanisms involved. The data generated can give significant insight into the immune mechanisms behind these vaccinations, which could lead to improved flavivirus vaccination strategies as well as provide potential protection against Zika virus while its vaccine is still in development. The data generated herein contributes to our understanding of flavivirus vaccines and leads to a potential novel vaccination method.

### EXAMPLE 2: ZIKA VIRUS-SPECIFIC T CELL RESPONSES GENERATED BY THE YELLOW FEVER VACCINE

On Feb. 1, 2016, the WHO declared that the reported clusters of microcephaly and other neurological disorders constituted a Public Health Emergency of International Concern (PHEIC). The Zika virus epidemic represents an unprecedented health crisis affecting significant parts of the world³². The epidemic is currently ongoing in Latin America and the Caribbean, and impacts of the infection are already seen in large populations in Brazil, Colombia, Mexico, Peru and beyond. The infection currently stands without specific treatment or a vaccine, although several vaccine candidates currently are under development³³.

The Zika virus is a mosquito borne virus belonging to the group of flaviviruses (family Flaviviridae), closely related to, e.g., the yellow fever virus (YFV). In 1939, Max Theiler succeeded in attenuating the YFV. Soon thereafter, a vaccine towards the YFV was developed and distributed worldwide and, to date, over 300 million doses of the vaccine have been administered to humans³⁴. Indeed, the YFV vaccine is still considered as one of the world's most efficient vaccines, where a single dose gives an at least 10-year (or most likely lifelong) immunity to the infection³⁵. Herein, we report that the YFV vaccine generate cross reactive CD4 and CD8 T cell responses to Zika virus antigens. The responses detected are mounted towards the NS5-protein of Zika virus.

The NS5-protein is a multifunctional, conserved protein in flaviviruses that constitutes the viral polymerase. By comparative analysis of NS5 protein sequences of the currently used live attenuated YFV vaccine and Zika virus³⁶, we found the yellow fever vaccine-derived NS5 protein to have 64% homology with Zika virus NS5. This led us to hypothesize that cross-reactive responses may occur between T cells obtained following YFV vaccination and Zika virus specific antigens. To test this, peripheral blood was collected from two Zika virus naïve donors before (day 0) and 15 days after (day 15) administration of the YFV vaccine. Cells were stimulated (according standard methods,³⁷) with a Zika-NS5 overlapping peptide library (Zika virus French polynesia isolate, 18 aminoacids in length and 10 aminoacids overlapping; library from GenScript) for 12 hours. Zika virus-specific T cells were subsequently identified as lymphocytes, singlets, dump- (CD19, CD14 and dead cell marker), CD3+CD4+/CD3+CD8+ and IFNγ+TNF+ cells. CD4 T cells producing both IFN-γ and TNF in response to the Zika-NS5 library were clearly increased at 15 days after YFV vaccination. Likewise, CD8 T cells producing both IFN-γ and TNF in response to the Zika-NS5 library were clearly increased at 15 days after YFV vaccination (see FIG. 8). These results indicate that the homology between the YFV vaccine and Zika virus-NS5 is high enough to induce Zika-specific T cells of both CD4 and CD8 lineage.

Neutralizing antibodies produced by B cells, are critical for vaccine-mediated protection against viral diseases. Cross-reactive antibodies among flaviviruses have been reported previously, and it is currently debated whether these are protective or contribute to pathogenesis^{38,39}. In contrast, cross-reactive T cell-mediated immunity amongst flaviviruses has been less well studied. It was recently demonstrated that the vaccine against Japanese encephalitis generates T cells that are cross-reactive with Dengue virus and West Nile virus⁴⁰. The present findings indicate that the YFV vaccine generates cell-mediated immune responses against Zika virus.

### EXAMPLE 3: SYNERGY IN RESPONSES INDUCED BY CONCOMITANT YFV AND TBEV VACCINATION

Methodology: Blood from an initial 20 individuals, immunized with YFV vaccine (Day 0) and TBE (Day 0+28) vaccines simultaneously, are collected before and at several time points after vaccination (FIG. 9). Individuals immunized with YFV and TBE vaccines alone are used as control groups. The immune responses are determined with several different approaches.

We have previously identified epitopes in both YFV and TBEV^{10,31}, and these are utilized to (i) study the phenotype of the TBE and YFV specific T cells (including markers for CD3, CD8, CD4, CD28, CTLA-4, CCR5, CD127, T-bet, Eomes, CD45RA, Ki67, CD69, perforin, granzyme B and CD38) and (ii), study T cells functions induced by the vaccines including markers for degranulation (CD107a), as well as cytokines and chemokines (TNF, IFN-γ, MIP-1β and IL-2).

The kinetics and magnitude of plasmablast appearance after vaccination are studied with multicolor flow cytometry. Phenotypical assessment over time is performed with markers for CD20, intracellular IgG, Ki67, PD-1, HLA-DR and CD80. Our pilot experiments have shown that plasmablasts are readily detectable following booster immunization to TBEV, indicating the feasibility of this approach (FIG. 3).

Elispot is used to functionally verify that the plasmablasts detected are vaccine-specific. Such methods are currently established in our lab (see Jahnimatz et al, 2013, J Immunol Methods, 391(1-2): 50-9).

Neutralizing antibodies are evaluated using plaque reduction neutralization tests²² and correlated with vaccine specific memory B cell and plasmablast numbers.

Multiplex assay on sera from all time points is performed, and inflammatory cytokines together with innate and adaptive cytokines will be measured.

### EXAMPLE 4: SYNERGY IN RESPONSES INDUCED BY CONCOMITANT YFV AND JEV VACCINATION

Methodology: Blood from 20 individuals, immunized with YFV (Day 0) and JEV (Day 0+28) vaccines simultaneously, is collected before and at several time points after vaccination (with the same collection schedule as in Example 3, FIG. 1). Individuals immunized with YFV and JE vaccines alone are used as control groups. The immune responses are then determined with the same approaches as in Example 3.

The YFV vaccine is given at day 0 simultaneously with JEV vaccine and a booster JEV vaccine at day 28. Early innate mechanism activation is evaluated with the same approaches as described in Example 3.

### EXAMPLE 5: CROSS REACTIVITY OF YFV VACCINATION TO ZIKV

Methodology: We have collected a cohort of healthy volunteers that were vaccinated against yellow fever virus. PBMC and sera have been collected before and at days, 10, 15 and 90 after administration of the vaccine. To measure possible cross-reactive T cells, we have generated overlapping peptide libraries of the conserved NS5 region of ZIKV and also TBE viruses.

The overlapping peptide libraries of ZIKV and TBEV are used to activate T cells in the YFV vaccinated donors.

The responses induced by ZIKV and TBEV peptides in the YFV vaccinated individuals are studied by flow cytometry. We will have markers for CD4 (T helper) and CD8 (cytotoxic T cells) together with classical markers for T cell function (IFN-γ, MIP-1β, IL-2).

HLA class I tetramers are generated for selected epitopes to study the appearance, magnitude and phenotype of cross-reactive T cells.

NS5 sequences
YFV asibi
(SEQ ID NO: 36)
VSRGTAKLRWFHERGYVKLEGRVIDLGCGRGGWCYYAAAQKEVSGVKGFT
LGRDGHEKPMNVQSLGWNIITFKDKTDIHRLEPVKCDTLLCDIGESSSSS
VTEGERTVRVLDTVEKWLACGVDNFCVKVLAPYMPDVLEKLELLQRRFGG
TVIRNPLSRNSTHEMYYVSGARTLEADVILPIGTRSVETDKGPLDKEATE
ERVERIKSEYMTSWEYDNDNPYRTWHYCGSYVTKTSGSAASMVNGVIKIL
TYPWDRIEEVTRMAMTDTTPFGQQRVFKEKVDTRAKDPPAGTRKIMKVVN
RWLFRHLAREKNPRLCTKEEFIAKVRSHAAIGAYLEEQEQWKTANEAVQD
PKFWELVDEERKLHQQGRCRTCVYNMMGKREKKLSEFGKAKGSRAIWYMW
LGARYLEFEALGFLNEDHWASRENSGGGVEGIGLQYLGYVIRDLAAMDGG
GFYADDTAGWDTRITEADLDDEQEILNYMSPHHKKLAQAVMEMTYKNKVV
KVLRPAPGGKAYMDVISRRDQRGSGQVVTYALNTITNLKVQLIRMAEAEM
VIHHQHVQDCDESVLTRLEAWLTEHGCNRLKRMAVSGDDCVVRPIDDRFG
LALSHLNAMSKVRKDISEWQPSKGWNDWENVPFCSHHFHELQLKDGRRIV
VPCREQDELIGRGRVSPGNGWMIKETACLSKAYANMWSLMYFHKRDMRLL
SLAVSSAVPTSWVPQGRTTWSIHGKGEWMTTEDMLEVWNRVWITNNPHMQ
DKTMVKEWRDVPYLTKRQDKLCGSLIGMTNRATWASHIHLVIHRIRTLIG
QEKYTDYLTVMDRYSVDADLQ
YFV 17DD (YF VAX)
(SEQ ID NO: 37)
VSRGTAKLRWFHERGYVKLEGRVIDLGCGRGGWCYYAAAQKEVSGVKGFT
LGRDGHEKPMNVQSLGWNIITFKDKTDIHRLEPVKCDTLLCDIGESSSSS
ITEGERTVRVLDTVEKWLACGVDNFCVKVLAPYMPDVLEKLELLQRRFGG
TVIRNPLSRNSTHEMYYVSGARTLEADVILPIGTRSVETDKGPLDKEATE
ERVERIKSEYMTSWEYDNDNPYRTWHYCGSYVTKTSGSAASMVNGVIKIL
TYPWDRIEEVTRMAMTDTTPFGQQRVFKEKVDTRAKDPPAGTRKIMKVVN
RWLFRHLAREKSPRLCTKEEFIAKVRSHAAIGAYLEEQEQWKTANEAVQD
PKFWELVDEERKLHQQGRCRTCVYNMMGKREKKLSEFGKAKGSRAIWYMW
LGARYLEFEALGFLNEDHWASRENSGGGVEGIGLQYLGYVIRDLAAMDGG
GFYADDTAGWDTRITEADLDDEQEILNYMSPHHKKLAQAVMEMTYKNKVV
KVLRPAPGGKAYMDVISRRDQRGSGQVVTYALNTITNLKVQLIRMAEAEN
VIHHQHVQDCDESVLTRLEAWLTEHGCNRLKRMAVSGDDCVVRPIDDRFG
LALSHLNAMSKVRKDISEWQPSKGWNDWENVPFCSHRFHELQLKDGRRIV
VPCREQDELIGRGRVSPGNGWMIKETACLSKAYANMWSLMYFHKRDMRLL
SLAVSSAVPTSWVPQGRTTWSIHGKGEWMTTEDMLEVWNRVWITNNPHMQ
DKTMVKKWRDVPYLTKRQDKLCGSLIGMTNRATWASHIHLVIHRIRTLIG
QEKYTDYLTVMDRYSVDADLQ
YFV 17D204 (Stamaril)
(SEQ ID NO: 38)
VSRGTAKLRWFHERGYVKLEGRVIDLGCGRGGWCYYAAAQKEVSGVKGFT
LGRDGHEKPMNVQSLGWNIITFKDKTDIHRLEPVKCDTLLCDIGESSSSS
VTEGERTVRVLDTVEKWLACGVDNFCVKVLAPYMPDVLEKLELLQRRFGG
TVIRNPLSRNSTHEMYYVSGARTLEADVILPIGTRSVETDKGPLDKEATE
ERVERIKSEYMTSWEYDNDNPYRTWHYCGSYVTKTSGSAASMVNGVIKIL
TYPWDRIEEVTRMAMTDTTPFGQQRVFKEKVDTRAKDPPAGTRKIMKVVN
RWLFRHLAREKNPRLCTKEEFIAKVRSHAAIGAYLEEQEQWKTANEAVQD
PKFWELVDEERKLHQQGRCRTCVYNMMGKREKKLSEFGKAKGSRAIWYMW
LGARYLEFEALGELNEDHWASRENSGGGVEGIGLQYLGYVIRDLAAMDGG
GFYADDTAGWDTRITEADLDDEQEILNYMSPHHKKLAQAVMEMTYKNKVV
KVLRPAPGGKAYMDVISRRDQRGSGQVVTYALNTITNLKVQLIRMAEAEM
VIHHQHVQDCDESVLTRLEAWLTEHGCNRLKRMAVSGDDCVVRPIDDRFG
LALSHLNAMSKVRKDISEWQPSKGWNDWENVPFCSHHFHELQLKDGRRIV
VPCREQDELIGRGRVSPGNGWMIKETACLSKAYANMWSLMYFHKRDMRLL
SLAVSSAVPTSWVPQGRTTWSIHGKGEWMTTEDMLEVWNRVWITNNPHMQ
DKTMVKKWRDVPYLTKRQDKLCGSLIGMTNRATWASHIHLVIHRIRTLIG
QEKYTDYLTVMDRYSVDADLQ

### EXAMPLE 6: CROSS REACTIVITY OF YFV VACCINATION TO JEV

### Methodology:

For mapping of T cell epitopes, freshly isolated PBMCs from one previously JEV-negative donor receiving the YFV vaccine, were incubated with JEV NS5 peptide library (18mer peptides, 70% pure, overlapping the NS5 protein of JEV) for 12 hours in the presence of brefeldin A and monensin (Sigma-Aldrich).

For phenotypic analysis, cells were incubated for 30 min at 4° C. in the dark with surface mAbs. For intracellular staining of TNF and IFN-γ, cells were fixed and permeabilized with Fix/Perm (eBioscience) for 30 min at 4° C. in the dark.

The following mAbs were used in flow cytometry: anti-CD4 Brilliant ultraviolet 737, anti-CD8 Brilliant ultraviolet 395, anti-CD3 PE-Cy5, anti-TNF Pacific Blue, anti-IFN-γ Brilliant Violet 785, Near IR, anti CD19 V500, anti CD14 V500. Flow cytometry data were acquired on a BD LSRFortessa (BD Biosciences) and analysed.

### Results:

FIG. 11 shows CD8 T cells from one donor receiving the YVF vaccine (donor By4), before (day 0) and at 15 days after vaccination. Production of TNF increases from 0.03% to 0.3% that is a 10 fold increase in response to the JEV NS5 peptide library.

### Conclusions

FIG. 11 demonstrates that JEV-specific T cells, able to produce TNF in response to JEV peptides, arise when given the YFV vaccine alone.

### REFERENCES

1 Organization, W. H. Japanese Encephalitis, <http://www.who.int/mediacentre/factsheets/fs386/en/> (2015).
2 Organization, W. H. Yellow fever, <http://www.who.int/mediacentre/factsheets/fs100/en/> (2014).
3 Organization, W. H. Dengue and severe dengue, <http://www.who.int/mediacentre/factsheets/fs117/en/> (2016).
4 Lindquist, L. & Vapalahti, O. Tick-borne encephalitis. The Lancet 371, 1861-1871, doi:10.1016/50140-6736(08)60800-4 (2008).
5 Organization, W. H. Zika virus, <http://www.who.int/mediacentre/factsheets/zika/en/> (2016).
6 Ahmed, R. & Akondy, R. S. Insights into human CD8(+) T-cell memory using the yellow fever and smallpox vaccines. Immunol Cell Biol 89, 340-345, doi:10.1038/icb.2010.155 (2011).
7 Pulendran, B. Learning immunology from the yellow fever vaccine: innate immunity to systems vaccinology. Nat Rev Immunol 9, 741-747, doi:10.1038/nri2629 (2009).
8 Miller, J. D. et al. Human effector and memory CD8+ T cell responses to smallpox and yellow fever vaccines. Immunity 28, 710-722, doi:10.1016/j.immuni.2008.02.020 (2008).
9 Akondy, R. S. et al. The yellow fever virus vaccine induces a broad and polyfunctional human memory CD8+ T cell response. J Immunol 183, 7919-7930, doi:10.4049/jimmunol.0803903 (2009).
10 Blom, K. et al. Temporal dynamics of the primary human T cell response to yellow fever virus 17D as it matures from an effector- to a memory-type response. J Immunol 190, 2150-2158, doi:10.4049/jimmunol. 1202234 (2013).
11 Marquardt, N. et al. The Human NK Cell Response to Yellow Fever Virus 17D Is Primarily Governed by NK Cell Differentiation Independently of NK Cell Education. J Immunol 195, 3262-3272, doi:10.4049/jimmunol.1401811 (2015).
12 Andersson, C. R. et al. Vaccine failures after active immunisation against tick-borne encephalitis. Vaccine 28, 2827-2831, doi:10.1016/j.vaccine.2010.02.001 (2010).
13 Haglund, M. & Gunther, G. Tick-borne encephalitis—pathogenesis, clinical course and long-term follow-up. Vaccine 21 Suppl 1, S11-18 (2003).
14 Donoso Mantke, O., Schadler, R. & Niedrig, M. A survey on cases of tick-borne encephalitis in European countries. Euro Surveill 13 (2008).
15 Halstead, S. B. Dengue Antibody-Dependent Enhancement: Knowns and Unknowns. Microbiol Spectr 2, doi:10.1128/microbiolspec.AID-0022-2014 (2014).
16 Mansfield, K. L. et al. Flavivirus-induced antibody cross-reactivity. J Gen Virol 92, 2821-2829, doi:10.1099/vir.0.031641-0 (2011).
17 Dejnirattisai, W. et al. Dengue virus sero-cross-reactivity drives antibody-dependent enhancement of infection with zika virus. Nat Immunol, doi:10.1038/ni.3515 (2016).
18 Hoke, C. H. et al. Protection against Japanese encephalitis by inactivated vaccines. N Engl J Med 319, 608-614, doi:10.1056/NEJM198809083191004 (1988).
19 Turtle, L. et al. Human T cell responses to Japanese encephalitis virus in health and disease. J Exp Med, doi:10.1084/jem.20151517 (2016).
20 Turtle, L. et al. Cellular Immune Responses to Live Attenuated Japanese Encephalitis (JE) Vaccine SA14-14-2 in Adults in a JE/Dengue Co-Endemic Area. PLoS Negl Trop Dis 11, e0005263, doi:10.1371/journal.pntd.0005263 (2017).
21 Identifying Candidate Targets of Immune Responses in Zika Virus Based on Homology to Epitopes in Other Flavivirus Species, <http://currents.plos.orq/outbreaks/article/identifyinq-candidate-tamets-of-immune-responses-in-zika-virus-based-on-homology-to-epitopes-in-other-flavivirus-species/> (2016).
22 Vene, S., Haglund, M., Vapalahti, O. & Lundkvist, A. A rapid fluorescent focus inhibition test for detection of neutralizing antibodies to tick-borne encephalitis virus. J Virol Methods 73, 71-75 (1998).
23 Querec, T. D. et al. Systems biology approach predicts immunogenicity of the yellow fever vaccine in humans. Nat Immunol 10, 116-125, doi:10.1038/ni.1688 (2009)
24 Wrammert, J. et al. Rapid and massive virus-specific plasmablast responses during acute dengue virus infection in humans. J Virol 86, 2911-2918, doi:10.1128/JVI.06075-11 (2012).
25 Beuneu, H., Garcia, Z. & Bousso, P. Cutting edge: cognate CD4 help promotes recruitment of antigen-specific CD8 T cells around dendritic cells. J Immunol 177, 1406-1410 (2006).
26 Castellino, F. et al. Chemokines enhance immunity by guiding naive CD8+ T cells to sites of CD4+ T cell-dendritic cell interaction. Nature 440, 890-895, doi:10.1038/nature04651 (2006).
27 Kumamoto, Y., Mattei, L. M., Sellers, S., Payne, G. W. & Iwasaki, A. CD4+ T cells support cytotoxic T lymphocyte priming by controlling lymph node input. Proc Natl Acad Sci USA 108, 8749-8754, doi:10.1073/pnas.1100567108 (2011).
28 Nakanishi, Y., Lu, B., Gerard, C. & Iwasaki, A. CD8(+) T lymphocyte mobilization to virus-infected tissue requires CD4(+) T-cell help. Nature 462, 510-513, doi:10.1038/nature08511 (2009).
29 Strutt, T. M. et al. Memory CD4+ T cells induce innate responses independently of pathogen. Nat Med 16, 558-564, 551p following 564, doi:10.1038/nm.2142 (2010).
30 Romagnani, S. T-cell subsets (Th1 versus Th2). Ann Allergy Asthma Immunol 85, 9-18; quiz 18, 21, doi:10.1016/S1081-1206(10)62426-X (2000).
31 Blom, K. et al. Specificity and dynamics of effector and memory CD8 T cell responses in human tick-borne encephalitis virus infection. PLoS Pathog 11, e1004622, doi:10.1371/journal.ppat.1004622 (2015).
32. Gostin L O, Hodge J G, Jr. Zika virus and global health security. The Lancet Infectious Diseases; 16(10): 1099-100.
33. Vannice K S, Giersing B K, Kaslow D C, et al. Meeting Report: WHO consultation on considerations for regulatory expectations of Zika virus vaccines for use during an emergency. Vaccine 2016.
34. Plotkin S A, Orenstein W A, Offit P A. Vaccines: Elsevier/Saunders; 2012.
35. Yellow fever vaccination booster not needed. World Health Organization.
36. Baronti C, Piorkowski G, Charrel R N, Boubis L, Leparc-Goffart I, de Lamballerie X. Complete coding sequence of zika virus from a French polynesia outbreak in 2013. Genome Announc 2014; 2(3).
37. Blom K, Braun M, Pakalniene J, et al. Specificity and dynamics of effector and memory CD8 T cell responses in human tick-borne encephalitis virus infection. PLoS Pathog 2015; 11(1): e1004622.
38. Mansfield K L, Horton D L, Johnson N, et al. Flavivirus-induced antibody cross-reactivity. J Gen Virol 2011; 92(Pt 12): 2821-9.
39. Dejnirattisai W, Supasa P, Wongwiwat W, et al. Dengue virus sero-cross-reactivity drives antibody-dependent enhancement of infection with zika virus. Nat Immunol 2016.
40. Turtle L, Tatullo F, Bali T, et al. Cellular Immune Responses to Live Attenuated Japanese Encephalitis (JE) Vaccine SA14-14-2 in Adults in a JE/Dengue Co-Endemic Area. PLoS Negl Trop Dis 2017; 11(1): e0005263.
41. Jolles S, Sewell W A, Misbah S A. Clinical uses of intravenous immunoglobulin. Clin Exp Immunol 2005; 142(1): 1-11.
42. Vene S, Haglund M, Vapalahti O, Lundkvist A. A rapid fluorescent focus inhibition test for detection of neutralizing antibodies to tick-borne encephalitis virus. J Virol Methods 1998; 73(1): 71-5.
43. Kuivanen S, Hepojoki J, Vene S, Vaheri A, Vapalahti O. Identification of linear human B-cell epitopes of tick-borne encephalitis virus. Virol J 2014; 11: 115.
44. Brandler S, Tangy F. Vaccines in development against West Nile virus. Viruses 2013; 5(10): 2384-409.
45. Vannice K S, Giersing B K, Kaslow D C, et al. Meeting Report: WHO consultation on considerations for regulatory expectations of Zika virus vaccines for use during an emergency. Vaccine 2016.
46. Weissbach F H, Hirsch H H. Comparison of Two Commercial Tick-Borne Encephalitis Virus IgG Enzyme-Linked Immunosorbent Assays. Clin Vaccine Immunol 2015; 22(7): 754-60.

1. A vaccine composition comprising a Yellow Fever Virus vaccine, for use in vaccinating an individual against infection by a Flavivirus; wherein the Flavivirus is not Yellow Fever Virus, West Nile Virus or Dengue; and wherein the Yellow Fever Virus vaccine generates a cross-reactive immune response to the Flavivirus.

2. A vaccine composition for use according to claim 1 wherein the individual has not previously been infected with the Flavivirus and/or vaccinated against the Flavivirus.

5. A vaccine composition for use according to claim 1 wherein the individual has not previously been infected with Yellow Fever Virus and/or vaccinated with the Yellow Fever Virus vaccine.

6. A vaccine composition for use according to claim 1, wherein the individual has previously been infected with the Flavivirus and/or vaccinated against the Flavivirus.

7. A vaccine composition for use according to claim 1, wherein the individual has previously been infected with Yellow Fever Virus and/or vaccinated with the Yellow Fever Virus vaccine.

8. A vaccine composition for use according to claim 1, wherein the Flavivirus is one or more Flavivirus selected from the group consisting of: Zika Virus; Tick-Borne Encephalitis Virus; Japanese Encephalitis Virus; West Nile Virus; Saint Louis Encephalitis Virus; Omsk Haemorrhagic Fever Virus.

9. A vaccine composition according to claim 1, wherein vaccinating the individual against infection by the Flavivirus prevents and/or reduces a subsequent infection by the Flavivirus and/or reduces one or more disorder and/or condition associated with infection by the Flavivirus.

10. A vaccine composition according to claim 9 wherein the prevention and/or reduction is effective for a period of up to 1 year or 5 years or 10 years.

11. A vaccine composition according to claim 9 wherein the reduction is by 100%, or by 90%, or by 80%, or by 70%, or by 60%, or by 50%.

12. The vaccine according to claim 1, wherein vaccinating the individual against infection by the Flavivirus generates and/or increases immunity in the individual against the Flavivirus.

13. A vaccine according to claim 12, wherein immunity comprises: cellular immunity and/or adaptive immunity in the individual against the Flavivirus; or wherein cellular immunity comprises T cell activity in the individual against the Flavivirus; or wherein the T cell activity comprises CD4+ T cell activity and/or CDS+ T cell activity.

14. A vaccine composition for use, or a use, or a method, according to claim 13 wherein adaptive immunity comprises B cell activity and/or antibody activity in the individual against the Flavivirus.

15. The vaccine of claim 1 wherein the antibodies bind to SEQ ID NO: 32, SEQ ID No: 32 SEQ ID No: 33, and/or SEQ ID No: 34

16. A vaccine composition comprising a Yellow Fever Virus vaccine and one or more additional vaccine against a Flavivirus, for use in vaccinating an individual against infection by the Flavivirus; wherein the Flavivirus is not Yellow Fever Virus, West Nile Virus or Dengue and wherein the Yellow Fever Virus vaccine and the one or more additional vaccine against a Flavivirus generate a cross-reactive immune response to the Flavivirus

17. A vaccine composition according to claim 16, wherein the one or more additional vaccine is selected from the group consisting of: a Zika Virus vaccine; a Tick-Borne Encephalitis Virus vaccine; a Japanese Encephalitis Virus vaccine; a West Nile Virus vaccine; a Saint Louis Encephalitis Virus vaccine; an Omsk Haemorrhagic Fever Virus vaccine.

18. A vaccine according to claim 17 wherein the one or more vaccine is intended to treat the Flavivirus of the one or more vaccine.

19. A vaccine composition for according to claim 16, wherein the individual has not previously been infected with the Flavivirus and/or vaccinated against the Flavivirus.

20. A vaccine composition for according to claim 16, wherein the individual has not previously been infected with Yellow Fever Virus and/or vaccinated with the Yellow Fever Virus vaccine.

21. A vaccine composition for according to claim 16, wherein the individual has previously been infected with the Flavivirus and/or vaccinated against the Flavivirus.

22. A vaccine composition for according to claim 16, wherein the individual has previously been infected with Yellow Fever Virus and/or vaccinated with the Yellow Fever Virus vaccine.

23. A vaccine composition for according to claim 16, wherein the Flavivirus is one or more Flavivirus selected from the group consisting of: Zika Virus; Tick-Borne Encephalitis Virus; Japanese Encephalitis Virus; West Nile Virus; Saint Louis Encephalitis Virus; Omsk Haemorrhagic Fever Virus.

24. A vaccine composition for according to claim 16, wherein vaccinating the individual against infection by the Flavivirus prevents and/or reduces a subsequent infection by the Flavivirus and/or prevents and/or reduces one or more disorder and/or condition associated with infection by the Flavivirus.

25. A vaccine composition for use, or a use, or a method, according to claim 24, wherein the prevention and/or reduction is effective for a period of up to 1 year; or 5 years; or 10 years.

26. A vaccine composition for use, or a use, or a method, according to claim 24, wherein the reduction by 100%, or by 90%, or by 80%, or by 70%, or by 60%, or by 50%.

27. A vaccine composition for use, or a use, or a method, according to claim 16 wherein vaccinating the individual against infection by the Flavivirus generates and/or increases immunity in the individual against the Flavivirus.

28. A vaccine composition for use, or a use, or a method, according to claim 27, wherein immunity comprises cellular immunity and/or adaptive immunity in the individual against the Flavivirus or wherein cellular immunity comprises T cell activity in the individual against the Flavivirus.

29. A vaccine composition, according to claim 28, wherein the T cell activity comprises CD4+ T cell activity and/or CDS+ T cell activity.

30. A vaccine composition according to claim 29 wherein adaptive immunity comprises B cell activity and/or antibody activity in the individual against the Flavivirus.

31. The vaccine of claim 30 wherein the antibodies bind to SEQ ID NO: 32, SEQ ID No: 32 SEQ ID No: 33, and/or SEQ ID No: 34

32. The use of a Yellow Fever Vaccine as an adjuvant.

33. A use according to claim 32, wherein the Yellow Fever Vaccine is used as an adjuvant for a vaccine, for example an inactivated vaccine.

34. A vaccine composition according to claim 16 wherein the Yellow Fever Virus vaccine is a live vaccine, and is preferably a live attenuated vaccine.

35. A vaccine composition according to claim 1, wherein the Yellow Fever Virus vaccine comprises a Yellow Fever Virus non-structural protein, or a fragment, variant or derivative thereof.

36. A vaccine composition wherein the Yellow Fever Virus vaccine comprises a polynucleotide sequence encoding a Yellow Fever Virus non-structural protein, or a fragment, variant or derivative thereof.

37. A vaccine composition for use, or a use, or a method, according to claim 36 wherein the Yellow Fever Virus non-structural protein comprises the NS5 protein, or a fragment, variant or derivative thereof.

38. The vaccine of claim 37 wherein the NS5 protein or fragment is selected from SEQ ID No: 1, SEQ ID No:39, SEQ ID No: 40, SEQ ID No: 41, SEQ ID No: 42, SEQ ID No:43, SEQ ID No: 44, SEQ ID No: 45, SEQ ID No: 46, SEQ ID No: 47, SEQ ID No:48, SEQ ID No: 49, SEQ ID No:50, SEQ ID No: 51, SEQ ID No:52, SEQ ID No: 53, SEQ ID No: 54, SEQ ID No: 55, SEQ ID No: 56, SEQ ID No: 57, SEQ ID No: 58, SEQ ID No:59, SEQ ID No:60, SEQ ID No:61, SEQ ID No:62, SEQ ID No: 63, SEQ ID No: 64, SEQ ID No: 65, SEQ ID No: 66, SEQ ID No: 67, SEQ ID No: 68 and/or SEQ ID No:69.

39. The vaccine of claim 1 wherein the vaccine generates antibodies which bind to E protein and NS1.

40. The vaccine of claim 16 wherein the vaccine generates antibodies which bind to E protein and NS1.

41. The vaccine composition of claim 1 further comprising a pharmaceutically-acceptable excipient or diluent.

42. The vaccine composition of claim 16 further comprising a pharmaceutically-acceptable excipient or diluent.

43. The vaccine of claim 1 wherein the vaccine is an Asibi strain, 17-DD, 17D204 or TBEV NEudorfl, TBEV Sojifin, TBEV Fe-205, TBEV Senzhang, JEV Nakayama, JEV SA-14-14-2, ZIKV Polyesian Strain 2013.

44. The vaccine of claim 1 which binds e protein or NS1 with 100, 1000 or 1000 times affinity of any other molecule in the patient.

45. The vaccine of claim 16 wherein the vaccine is an Asibi strain, 17-DD, 17D204 or TBEV NEudorfl, TBEV Sojifin, TBEV Fe-205, TBEV Senzhang, JEV Nakayama, JEV SA-14-14-2, ZIKV Polyesian Strain 2013.

46. The vaccine of claim 16 which binds e protein or NS1 with 100, 1000 or 1000 times affinity of any other molecule in the patient.

47. A method for vaccinating an individual against infection by a Flavivirus, the method comprising the step of administering to the individual a vaccine composition comprising a Yellow Fever Virus vaccine; wherein the Flavivirus is not Yellow Fever Virus; and wherein the Yellow Fever Virus vaccine generates a cross-reactive immune response to the Flavivirus.

48. The method of claim 47 further comprising administering at least additional vaccine against a Flavivirus; wherein the Flavivirus is not Yellow Fever Virus.

49. The method according to claim 48, wherein the Yellow Fever Virus vaccine and the at least one additional vaccine against a Flavivirus are administered simultaneously to the individual.

50. The method according to claim 48 wherein the Yellow Fever Virus vaccine and the one or more additional vaccine against a Flavivirus are administered sequentially to the individual.


SEQUENCE LISTING (69 sequences)

SEQ ID NO: 1 - protein, 9 aa, Yellow fever virus
ETACLSKAY

SEQ ID NO: 2 - protein, 9 aa, Yellow fever virus
RTWAYHGSY

SEQ ID NO: 3 - protein, 9 aa, Yellow fever virus
ALNTFTNLV

SEQ ID NO: 4 - protein, 9 aa, Yellow fever virus
YMWLGARFL

SEQ ID NO: 5 - protein, 9 aa, Yellow fever virus
RTTWSIHGK

SEQ ID NO: 6 - protein, 9 aa, Yellow fever virus
CVYNMMGKR

SEQ ID NO: 7 - protein, 9 aa, Yellow fever virus
GLVRVPLSR

SEQ ID NO: 8 - protein, 9 aa, Yellow fever virus
YTYQNKVVK

SEQ ID NO: 9 - protein, 9 aa, Yellow fever virus
NMMGKREKK

SEQ ID NO: 10 - protein, 9 aa, Yellow fever virus
GLGLQRLGY

SEQ ID NO: 11 - protein, 9 aa, Yellow fever virus
VPCRHQDEL

SEQ ID NO: 12 - protein, 9 aa, Yellow fever virus
GLQRLGYVL

SEQ ID NO: 13 - protein, 9 aa, Yellow fever virus
WLGARFLEF

SEQ ID NO: 14 - protein, 9 aa, Yellow fever virus
LLYFHRRDL

SEQ ID NO: 15 - protein, 9 aa, Yellow fever virus
SGQVVTYAL

SEQ ID NO: 16 - protein, 9 aa, Yellow fever virus
STLNGGLFY

SEQ ID NO: 17 - protein, 9 aa, Yellow fever virus
ETACLSKAY

SEQ ID NO: 18 - protein, 9 aa, Yellow fever virus
GVEGISLNY

SEQ ID NO: 19 - protein, 9 aa, Yellow fever virus
VMEWRDVPY

SEQ ID NO: 20 - protein, 9 aa, Yellow fever virus
VLAPYRPEV

SEQ ID NO: 21 - protein, 9 aa, Yellow fever virus
YMWLGSRFL

SEQ ID NO: 22 - protein, 9 aa, Yellow fever virus
MLVSGDDCV

SEQ ID NO: 23 - protein, 9 aa, Yellow fever virus
YALNTLTNI

SEQ ID NO: 24 - protein, 9 aa, Yellow fever virus
TLTNIKVQL

SEQ ID NO: 25 - protein, 9 aa, Yellow fever virus
CVYNMMGKR

SEQ ID NO: 26 - protein, 9 aa, Yellow fever virus
KLGEFGVAK

SEQ ID NO: 27 - protein, 9 aa, Yellow fever virus
AKVKSNAAL

SEQ ID NO: 28 - protein, 9 aa, Yellow fever virus
VVTYALNTL

SEQ ID NO: 29 - protein, 9 aa, Yellow fever virus
IAKVKSNAA

SEQ ID NO: 30 - protein, 9 aa, Yellow fever virus
SGQVVTYAL

SEQ ID NO: 31 - protein, 17 aa, Yellow fever virus
ALNTLTNIKVQLIRMME

SEQ ID NO: 32 - protein, 18 aa, Yellow fever virus
ALNTLTNIKVQLIRMMEG

SEQ ID NO: 33 - protein, 18 aa, Yellow fever virus
GKALYFLNDMAKTRKDIG

SEQ ID NO: 34 - protein, 18 aa, Yellow fever virus
WSIHASGAWMTTEDMLDV

SEQ ID NO: 35 - protein, 18 aa, Yellow fever virus
PWLAWHVAANVSSVTDRS

SEQ ID NO: 36 - protein, 821 aa, Yellow fever virus
VSRGTAKLRWFHERGYVKLEGRVIDLGCGRGGWCYYAAAQKEVSGVKGFTLGRDGHEKPMNVQSLGWNIITFKDKTDIHRLEPVKCDTLLCDIGESSSSSVTEGERTVRVLDTVEKWLACGVDNFCVKVLAPYMPDVLEKLELLQRRFGGTVIRNPLSRNSTHEMYYVSGARTLEADVILPIGTRSVETDKGPLDKEAIEERVERIKSEYMTSWFYDNDNPYRTWHYCGSYVTKTSGSAASMVNGVIKILTYPWDRIEEVTRMAMTDTTPFGQQRVFKEKVDTRAKDPPAGTRKIMKVVNRWLFRHLAREKNPRLCTKEEFIAKVRSHAAIGAYLEEQEQWKTANEAVQDPKFWELVDEERKLHQQGRCRTCVYNMMGKREKKLSEFGKAKGSRAIWYMWLGARYLEFEALGFLNEDHWASRENSGGGVEGIGLQYLGYVIRDLAAMDGGGFYADDTAGWDTRITEADLDDEQEILNYMSPHHKKLAQAVMEMTYKNKVVKVLRPAPGGKAYMDVISRRDQRGSGQVVTYALNTITNLKVQLIRMAEAEMVIHHQHVQDCDESVLTRLEAWLTEHGCNRLKRMAVSGDDCVVRPIDDRFGLALSHLNAMSKVRKDISEWQPSKGWNDWENVPFCSHHFHELQLKDGRRIVVPCREQDELIGRGRVSPGNGWMIKETACLSKAYANMWSLMYFHKRDMRLLSLAVSSAVPTSWVPQGRTTWSIHGKGEWMTTEDMLEVWNRVWITNNPHMQDKTMVKEWRDVPYLTKRQDKLCGSLIGMTNRATWASHIHLVIHRIRTLIGQEKYTDYLTVMDRYSVDADLQ

SEQ ID NO: 37 - protein, 821 aa, Yellow fever virus
VSRGTAKLRWFHERGYVKLEGRVIDLGCGRGGWCYYAAAQKEVSGVKGFTLGRDGHEKPMNVQSLGWNIITFKDKTDIHRLEPVKCDTLLCDIGESSSSSITEGERTVRVLDTVEKWLACGVDNFCVKVLAPYMPDVLEKLELLQRRFGGTVIRNPLSRNSTHEMYYVSGARTLEADVILPIGTRSVETDKGPLDKEAIEERVERIKSEYMTSWFYDNDNPYRTWHYCGSYVTKTSGSAASMVNGVIKILTYPWDRIEEVTRMAMTDTTPFGQQRVFKEKVDTRAKDPPAGTRKIMKVVNRWLFRHLAREKSPRLCTKEEFIAKVRSHAAIGAYLEEQEQWKTANEAVQDPKFWELVDEERKLHQQGRCRTCVYNMMGKREKKLSEFGKAKGSRAIWYMWLGARYLEFEALGFLNEDHWASRENSGGGVEGIGLQYLGYVIRDLAAMDGGGFYADDTAGWDTRITEADLDDEQEILNYMSPHHKKLAQAVMEMTYKNKVVKVLRPAPGGKAYMDVISRRDQRGSGQVVTYALNTITNLKVQLIRMAEAENVIHHQHVQDCDESVLTRLEAWLTEHGCNRLKRMAVSGDDCVVRPIDDRFGLALSHLNAMSKVRKDISEWQPSKGWNDWENVPFCSHRFHELQLKDGRRIVVPCREQDELIGRGRVSPGNGWMIKETACLSKAYANMWSLMYFHKRDMRLLSLAVSSAVPTSWVPQGRTTWSIHGKGEWMTTEDMLEVWNRVWITNNPHMQDKTMVKKWRDVPYLTKRQDKLCGSLIGMTNRATWASHIHLVIHRIRTLIGQEKYTDYLTVMDRYSVDADLQ

SEQ ID NO: 38 - protein, 821 aa, Yellow fever virus
VSRGTAKLRWFHERGYVKLEGRVIDLGCGRGGWCYYAAAQKEVSGVKGFTLGRDGHEKPMNVQSLGWNIITFKDKTDIHRLEPVKCDTLLCDIGESSSSSVTEGERTVRVLDTVEKWLACGVDNFCVKVLAPYMPDVLEKLELLQRRFGGTVIRNPLSRNSTHEMYYVSGARTLEADVILPIGTRSVETDKGPLDKEAIEERVERIKSEYMTSWFYDNDNPYRTWHYCGSYVTKTSGSAASMVNGVIKILTYPWDRIEEVTRMAMTDTTPFGQQRVFKEKVDTRAKDPPAGTRKIMKVVNRWLFRHLAREKNPRLCTKEEFIAKVRSHAAIGAYLEEQEQWKTANEAVQDPKFWELVDEERKLHQQGRCRTCVYNMMGKREKKLSEFGKAKGSRAIWYMWLGARYLEFEALGFLNEDHWASRENSGGGVEGIGLQYLGYVIRDLAAMDGGGFYADDTAGWDTRITEADLDDEQEILNYMSPHHKKLAQAVMEMTYKNKVVKVLRPAPGGKAYMDVISRRDQRGSGQVVTYALNTITNLKVQLIRMAEAEMVIHHQHVQDCDESVLTRLEAWLTEHGCNRLKRMAVSGDDCVVRPIDDRFGLALSHLNAMSKVRKDISEWQPSKGWNDWENVPFCSHHFHELQLKDGRRIVVPCREQDELIGRGRVSPGNGWMIKETACLSKAYANMWSLMYFHKRDMRLLSLAVSSAVPTSWVPQGRTTWSIHGKGEWMTTEDMLEVWNRVWITNNPHMQDKTMVKKWRDVPYLTKRQDKLCGSLIGMTNRATWASHIHLVIHRIRTLIGQEKYTDYLTVMDRYSVDADLQ

SEQ ID NO: 39 - protein, 9 aa, Yellow fever virus
VLAPYMPDV

SEQ ID NO: 40 - protein, 9 aa, Yellow fever virus
YMPDVLEKL

SEQ ID NO: 41 - protein, 9 aa, Yellow fever virus
RNSTHEMYY

SEQ ID NO: 42 - protein, 9 aa, Yellow fever virus
RVERIKSEY

SEQ ID NO: 43 - protein, 9 aa, Yellow fever virus
WFYDNDNPY

SEQ ID NO: 44 - protein, 9 aa, Yellow fever virus
RTWHYCGSY

SEQ ID NO: 45 - protein, 9 aa, Yellow fever virus
MAMTDTTPF

SEQ ID NO: 46 - protein, 9 aa, Yellow fever virus
KVVNRWLFR

SEQ ID NO: 47 - protein, 9 aa, Yellow fever virus
RSHAAIGAY

SEQ ID NO: 48 - protein, 9 aa, Yellow fever virus
WLGARYLEF

SEQ ID NO: 49 - protein, 9 aa, Yellow fever virus
GVEGIGLQY

SEQ ID NO: 50 - protein, 9 aa, Yellow fever virus
AAMDGGGFY

SEQ ID NO: 51 - protein, 9 aa, Yellow fever virus
YMSPHHKKL

SEQ ID NO: 52 - protein, 9 aa, Yellow fever virus
RPAPGGKAY

SEQ ID NO: 53 - protein, 9 aa, Yellow fever virus
RPIDDRFGL

SEQ ID NO: 54 - protein, 9 aa, Yellow fever virus
YANMWSLMY

SEQ ID NO: 55 - protein, 9 aa, Yellow fever virus
YFHKRDMRL

SEQ ID NO: 56 - protein, 9 aa, Yellow fever virus
VKKWRDVPY

SEQ ID NO: 57 - protein, 9 aa, Yellow fever virus
RTLIGQEKY

SEQ ID NO: 58 - protein, 9 aa, Yellow fever virus
RSHAAIGAY

SEQ ID NO: 59 - protein, 9 aa, Yellow fever virus
TPFGQQRVF

SEQ ID NO: 60 - protein, 9 aa, Yellow fever virus
MWHVTRGAF

SEQ ID NO: 61 - protein, 9 aa, Yellow fever virus
SVKEDLVAY

SEQ ID NO: 62 - protein, 9 aa, Yellow fever virus
CARRRLRTL

SEQ ID NO: 63 - protein, 9 aa, Yellow fever virus
RRRLRTLVL

SEQ ID NO: 64 - protein, 9 aa, Yellow fever virus
DVKFHTQAF

SEQ ID NO: 65 - protein, 9 aa, Yellow fever virus
AMCHATLTY

SEQ ID NO: 66 - protein, 9 aa, Yellow fever virus
RANESATIL

SEQ ID NO: 67 - protein, 9 aa, Yellow fever virus
VVVLNRKTF

SEQ ID NO: 68 - protein, 9 aa, Yellow fever virus
RVLDCRTAF

SEQ ID NO: 69 - protein, 9 aa, Yellow fever virus
SMLLDNMEV